(19) United States
(12) Patent Application Publication
(10) Pub. No.: US 2025/0041403 A1
(43) Pub. Date: Feb. 6, 2025

(54) Physiological Metrics as Candidate Predictors of Antibody Response Following Vaccination Against Covid-19

(71) Applicants: Ashley E. MASON, San Francisco, CA (US); Frederick M. HECHT, San Francisco, CA (US); Benjamin SMARR, San Francisco, CA (US); Patrick KASL, San Francisco, CA (US); The Regents of the University of California, Oakland, CA (US); U.S. Government represented by the Department of Veterans Affairs, Washington, DC (US)

(72) Inventors: Ashley E. Mason, San Francisco, CA (US); Frederick M. Hecht, San Francisco, CA (US); Benjamin Smarr, San Francisco, CA (US); Patrick Kasl, San Francisco, CA (US)

(21) Appl. No.: 18/717,783

(22) PCT Filed: Dec. 9, 2022
(86) PCT No.: PCT/US2022/052356

§ 371 Date: Jun. 7, 2024

Related U.S. Application Data
(60) Provisional application No. 63/287,914, filed on Dec. 9, 2021.

(51) Int. Cl. A61K 39/215; A61B 5/0205; A61K 39/00; G01N 33/569

(52) U.S. Cl. CPC A61K 39/215; G01N 33/56983; A61B 5/02055; A61K 2039/53; G01N 2333/165; G01N 2469/20

(57) ABSTRACT

Methods, systems, and devices are provided for predicting the robustness of an antibody response of a subject to the SARS-COV-2 spike protein receptor binding domain based on measurement of physiological metrics following vaccination. In particular, one or more physiological metrics of the subject selected from dermal temperature deviation, heart rate, respiratory rate, heart rate variability, and deep sleep duration are measured with a wearable device before and after vaccinating the subject, wherein increases in dermal temperature deviation, heart rate, and respiratory rate, and decreases in heart rate variability on the first night after administration of the vaccine to the subject are correlated with greater antibody responses to the SARS-COV-2 spike protein.

### CROSS-REFERENCE TO RELATED APPLICATIONS

This application claims the benefit of U.S. Provisional Patent Application No. 63/287,914, filed Dec. 9, 2021, which application is incorporated herein by reference in its entirety.

### GOVERNMENT RIGHTS

This invention was made with government support under W81XWH-15-9-0001 awarded by the Medical Research and Development Command. The government has certain rights in the invention

### BACKGROUND OF THE INVENTION

There is significant variability among individuals in their ability to develop neutralizing antibody responses to coronavirus disease 2019 (COVID-19) vaccines, which correlate with immune protection. Thus, there is a need in the art to identify predictors of neutralizing antibody responses to vaccination and what physiological parameters may correlate with levels of antibodies to the severe acute respiratory syndrome coronavirus 2 (SARS-COV-2) receptor binding domain (RBD), the target of neutralizing antibodies generated by existing COVID-19 vaccines.

### SUMMARY OF THE INVENTION

Methods, systems, and devices are provided for predicting the robustness of an antibody response of a subject to the SARS-COV-2 spike protein receptor binding domain based on measurement of physiological metrics following vaccination. In particular, one or more physiological metrics of the subject selected from dermal temperature deviation, heart rate, respiratory rate, heart rate variability, and deep sleep duration are measured with a wearable device before and after vaccinating the subject, wherein increases in dermal temperature deviation, heart rate, and respiratory rate, and decreases in heart rate variability and deep sleep duration on the first night after administration of the vaccine to the subject are correlated with greater antibody responses to the SARS-COV-2 spike protein.

In one aspect, a method of determining whether vaccination of a subject against SARS-CoV-2 produces an antibody response to a spike protein receptor binding domain (RBD) of the SARS-COV-2 is provided, the method comprising: vaccinating the subject by administering a vaccine against the SARS-COV-2; and measuring one or more physiological metrics of the subject selected from dermal temperature deviation, heart rate, respiratory rate, heart rate variability, and deep sleep duration before and after said vaccinating the subject using a measuring device, wherein increases in the dermal temperature deviation, heart rate, and respiratory rate, and decreases in the heart rate variability and deep sleep duration on the first night after said vaccinating the subject compared to a pre-vaccination dermal temperature deviation, heart rate, respiratory rate, heart rate variability, and deep sleep duration of the subject correlate with increases in the antibody response of the subject to the SARS-COV-2 spike protein.

In certain embodiments, the vaccine comprises mRNA or DNA encoding the SARS-CoV-2 spike protein RBD.

In certain embodiments, the measuring comprises measuring two or more of the physiological metrics.

In certain embodiments, the measuring comprises measuring dermal temperature deviation, heart rate, respiratory rate, heart rate variability, and deep sleep duration.

In certain embodiments, the measuring comprises measuring dermal temperature deviation.

In certain embodiments, the vaccinating comprises administering at least a first dose of the vaccine and a second dose of the vaccine to the subject. In some embodiments, the measuring comprises measuring the one or more physiological metrics of the subject before and after the second dose of the vaccine is administered to the subject.

In certain embodiments, the vaccinating comprises administering the vaccine according to a prime-boost regimen. For example, one or more booster doses of the vaccine may be administered to the subject. In some embodiments, the measuring comprises measuring the one or more physiological metrics of the subject before and after a booster dose of the vaccine is administered to the subject.

In certain embodiments, the measuring device is portable.

In certain embodiments, the measuring device is a wearable device worn by the subject.

In certain embodiments, the measuring device comprises a tri-axial accelerometer.

In certain embodiments, the heart rate, respiratory rate, and heart rate variability are measured from a photoplethysmogram (PPG) signal.

In certain embodiments, the dermal temperature is measured using a negative temperature coefficient (NTC) thermistor. For example, dermal temperature readings may be acquired from the base of a finger on the palm side of a hand of the subject.

In certain embodiments, the increases in the dermal temperature deviation, heart rate, and respiratory rate, and decreases in the heart rate variability and deep sleep duration on the first night following said vaccinating the subject are correlated with increases in the antibody response to the SARS-COV-2 spike protein using a Spearman rank-order correlation analysis or a Kendall rank order correlation analysis.

In certain embodiments, the physiological metrics are measured during sleep periods, wake periods, or both sleep and wake periods of the subject.

In certain embodiments, the method further comprises measuring the one or more physiological metrics of the subject for at least one or two months prior to vaccination of the subject to determine average baseline values for the one or more physiological metrics, wherein increases in dermal temperature deviation, heart rate, and respiratory rate, and decreases in heart rate variability on the first night after said vaccinating the subject compared to the average baseline values for the dermal temperature deviation, heart rate, respiratory rate, heart rate variability, and deep sleep duration are correlated with increases in the antibody response of the subject to the SARS-COV-2 spike protein.

In certain embodiments, the method further comprises measuring average overnight dermal temperature of the subject for at least one or two months prior to vaccination of the subject to determine an average baseline value for the overnight dermal temperature, wherein the temperature deviation is computed as a difference between an average overnight temperature of the subject and the average baseline value of the overnight temperature.

In certain embodiments, the method further comprises measuring a serum level of antibodies to the SARS-COV-2 spike protein RBD for the subject if the dermal temperature deviation, heart rate, respiratory rate, heart rate variability, and deep sleep duration on the first night after said vaccinating the subject indicate that the level of antibodies to the SARS-COV-2 spike protein RBD is below a threshold level for effective neutralization of SARS-COV-2.

In certain embodiments, the method further comprises administering another booster dose of the vaccine to the subject if the one or more physiological metrics of the subject indicate that the level of antibodies to the SARS-COV-2 spike protein RBD is below a threshold level for effective neutralization of SARS-COV-2.

In another aspect, a system for determining whether vaccination of a subject against SARS-COV-2 produces an antibody response to a spike protein RBD of the SARS-COV-2 is provided, the system comprising: a) a measuring device configured to measure one or more physiological metrics of the subject selected from dermal temperature deviation, heart rate, respiratory rate, heart rate variability, and deep sleep duration before and after vaccinating the subject against SARS-COV-2; b) a processor programmed to analyze the physiological metrics using one or more algorithms to correlate increases in the dermal temperature deviation, heart rate, and respiratory rate, and decreases in the heart rate variability and deep sleep duration on the first night after said vaccinating the subject compared to a pre-vaccination dermal temperature deviation, heart rate, respiratory rate, heart rate variability, and deep sleep duration with increases in the antibody response of the subject to the SARS-COV-2 spike protein RBD; and c) an output device configured to display information regarding the antibody response of the subject to the SARS-COV-2 spike protein RBD.

In certain embodiments, the system further comprises a storage component operably coupled to the measuring device and the processor, wherein the storage component is configured to record physiological metric data measured by the measuring device.

In certain embodiments, the one or more algorithms are selected from a Spearman rank-order correlation analysis and a Kendall rank order correlation analysis.

In certain embodiments, the measuring device comprises a negative temperature coefficient (NTC) thermistor.

In certain embodiments, the measuring device comprises a tri-axial accelerometer.

In certain embodiments, the measuring device measures heart rate, respiratory rate, and heart rate variability from a photoplethysmogram (PPG) signal.

In certain embodiments, the measuring device of the system is portable.

In certain embodiments, the measuring device of the system is a wearable device worn by the subject.

In another aspect, a kit is provided, the kit comprising a system described herein, packaging for the system, and instructions for using the system for determining whether vaccination of a subject against severe acute respiratory syndrome coronavirus 2 (SARS-COV-2) produces an antibody response to a spike protein receptor binding domain (RBD) of the SARS-CoV-2.

### BRIEF DESCRIPTION OF THE DRAWINGS

FIG. 1. Participant flow through the study.

FIGS. 2A-2D. Plots depicting [FIG. 2A] changes in heart rate (HR), [FIG. 2B] heart rate variability (HRV), [FIG. 2C] respiration rate (RR), and [FIG. 2D] temperature deviation the nights surrounding the second injection for Pfizer-BioNTech and Moderna-NAIAD vaccine recipients, combined. Values are z-scored for participants' pre-vaccination baseline period (see Methods).

### DETAILED DESCRIPTION

Methods, systems, and devices are provided for predicting the robustness of an antibody response of a subject to the SARS-COV-2 spike protein receptor binding domain based on measurement of physiological metrics following vaccination. In particular, one or more physiological metrics of the subject selected from dermal temperature deviation, heart rate, respiratory rate, heart rate variability, and deep sleep duration are measured with a wearable device before and after vaccinating the subject, wherein increases in dermal temperature deviation, heart rate, and respiratory rate, and decreases in heart rate variability and deep sleep duration on the first night after administration of the vaccine to the subject are correlated with greater antibody responses to the SARS-COV-2 spike protein.

Before the present methods, systems, and devices are described, it is to be understood that this invention is not limited to particular methods, devices, or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a virus” includes a plurality of such viruses, and reference to “the antibody” includes reference to one or more antibodies and equivalents thereof, such as immunoglobulins, and the like, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

An “immunological response” or “immune response” to an antigen or composition is the development in a subject of a humoral and/or a cellular immune response to an antigen present in a composition of interest. For purposes of the present disclosure, a “humoral immune response” refers to an immune response mediated by antibody molecules, while a “cellular immune response” is one mediated by T-lymphocytes and/or other white blood cells. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γδ T-cells directed specifically to an antigen or antigens present in a composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “antibody response” refers herein to a humoral response of a subject to an antigen, including antibody-mediated immunity. An “antibody response to SARS-COV-2” refers herein to an antibody response resulting from vaccination against SARS-COV-2 that produces antibodies that bind to the SARS-COV-2 spike protein receptor binding domain.

The term “baseline” refers herein to an initial value measured or a known standard value for a physiological metric of a subject before vaccination or exposure to SARS-COV-2. In some embodiments, the baseline value or range of values of a physiological metric of a subject may be measured during a pre-vaccination baseline period. In some embodiments, the baseline period ranges from 1 week to 2 months or more before vaccination, or any period of time in between this range. For example, the baseline period may be 2 months, 1.5 months, 1 month, 3 weeks, 2 weeks, or 1 week before vaccination. A baseline physiological metric value may be subject-specific and used for comparison of physiological metric values obtained after vaccination or as a control for the subject.

The terms “connected” or “coupled” are used in an operational sense and are not necessarily limited to a direct connection or coupling. For example, two devices or components may be coupled directly, or via one or more intermediary media or devices. As another example, devices may be coupled in such a way that information or data can be passed between them, while not sharing any physical connection with one another. In some cases, two devices or components may be connected by a wire or wirelessly to each other.

The terms “individual”, “subject”, and “patient” are used interchangeably herein and include any mammalian subject for whom diagnosis, treatment, or therapy is desired, such as humans. “Mammal” refers to any animal classified as a mammal, including human and non-human primates, domestic animals and farm animals, zoo animals, animals used in sports, pets, and animals used in research, such as non-human primates, dogs, horses, cats, cows, sheep, goats, pigs, camels, rodents, etc.

### Methods and Systems for Evaluating Antibody Responses to the SARS-COV-2 Spike Protein

As discussed above, the methods and systems described herein provide a novel approach to detecting and/or predicting the robustness of an antibody response of a subject to the SARS-COV-2 spike protein receptor binding domain based on measurement of physiological metrics following vaccination. Antibody responses can be predicted based on a unique signature of changes in physiological metrics that currently available measuring devices are capable of measuring. Such physiological metrics include, without limitation, dermal temperature deviation, heart rate, respiratory rate, heart rate variability, and deep sleep duration. Increases in dermal temperature deviation, heart rate, and respiratory rate, and decreases in heart rate variability and deep sleep duration of a subject on the first night after vaccination compared to their pre-vaccination values can be correlated with greater antibody responses to the SARS-COV-2 spike protein receptor binding domain.

In certain embodiments, one or more physiological metrics selected from dermal temperature deviation, heart rate, respiratory rate, heart rate variability, and deep sleep duration are measured using a measuring device before and after vaccinating a subject against SARS-COV-2. In certain embodiments, 1 physiological metric, 1 to 2 physiological metrics, 2 to 3 physiological metrics, 3 to 4 physiological metrics, or 4 to 5 physiological metrics are measured. In one embodiment, dermal temperature deviation, heart rate, respiratory rate, heart rate variability, and deep sleep duration are measured. In another embodiment, dermal temperature deviation is measured.

Any suitable measuring device or combination of measuring devices may be used to measure the physiological metrics of the subject. The measuring device is preferably portable, or more preferably, a wearable device that can be readily used to monitor and measure the physiological metrics of the subject. For example, a portable or wearable device can be carried or worn by a subject outside of a clinical setting, such as at home or at work. Wearable measurement devices may be worn on a finger, wrist, arm, thigh, ankle, foot, chest, head, or other body part. Suitable measuring devices are commercially available, for example, from Oura Health Oy (Oulu, FI), Blue Spark Technologies, Inc. (Westlake, OH), Apple Inc. (Cupertino, CA), Fitbit (San Francisco, CA), Garmin Ltd. (Olathe, KS), NordicTrack (Logan, Utah), Omron Healthcare, Phillips (Amsterdam, Netherlands), Biostrap (Los Angeles, CA), Shimmer Sensing (Dublin, Ireland), and ActiGraph (Pensacola, FL).

In one embodiment, a wearable measuring device is used comprising a ring that can be worn by a subject on a finger. In some embodiments, the ring is sized to be suitably worn on a particular finger of the subject's choosing. Further, the ring may be available in a variety of sizes for accommodating various finger sizes of different users.

In another embodiment, a wearable measuring device is used comprises a wrist band that may be worn on a wrist of the subject. The wrist band of the measuring device should fit and be large enough to be suitably worn on the wrist of the subject. Further, the wrist band may be available in a variety of sizes for accommodating various wrist sizes for different users. In some instances, the wearable measuring device may be a smart watch comprising one or more sensors capable of monitoring physiological metrics of the subject.

In another embodiment, a wearable measuring device is used comprising a patch that can be attached to the surface of the skin on a suitable part of the body of a subject for measuring the physiological metrics such as, but not limited to, the arm, neck, wrist, finger, thigh, ankle, foot, chest, head, or other body part. The wearable patch may contain, for example, a foam, film, or cloth embedded with sensor electronics, which can be applied to the skin with an adhesive. In some instances, multiple patches may be used, each comprising different sensors, which may be placed at different positions on the body for measuring different physiological metrics of the subject.

In another embodiment, the measuring device is integrated into the fabric of a garment that can be worn by the subject. The garment carrying the measuring device should fit and be large enough to be suitably worn by the subject. Further, the garment may be available in a variety of sizes for accommodating various users of different sizes. In some instances, the garment may comprise multiple sensors at different positions for measuring different physiological metrics of the subject.

A single measuring electronic device may be capable of measuring one or more physiological metrics of the subject. For example, the measuring device may include, without limitation, a photoplethysmography (PPG) sensor, an accelerometer, a temperature sensor, a cardiac sensor, an infrared transmitter, an infrared receiver, a microcontroller, a radio frequency transceiver, and the like. In certain embodiments, the heart rate, respiratory rate, and heart rate variability are measured from a photoplethysmogram (PPG) signal. In certain embodiments, the dermal temperature is measured using a negative temperature coefficient (NTC) thermistor. For example, dermal temperature readings may be acquired from the base of a finger on the palm side of a hand of the subject. Movement and cardiac sensors can be used to track deep sleep. The device may comprise an accelerometer to measure movement of the subject or motion of parts of the body. In some embodiments, the measuring device comprises a tri-axial accelerometer to track movements in three orthogonal directions.

In certain embodiments, one or more of the physiological metrics of the subject are monitored continuously or intermittently over a pre-vaccination period or baseline period to establish a baseline range of values for the physiological metrics. In some embodiments, the baseline period ranges from 1 week to 2 months or more before vaccination, or any period of time in between this range. For example, the baseline period may be 2 months, 1.5 months, 1 month, 3 weeks, 2 weeks, or 1 week before vaccination. A baseline physiological metric value may be subject-specific and used for comparison of physiological metric values obtained after vaccination or as a control for the subject. For example, increases in dermal temperature deviation, heart rate, and respiratory rate, and decreases in heart rate variability and deep sleep duration on the first night after said vaccinating the subject compared to the average baseline values for the dermal temperature deviation, heart rate, respiratory rate, heart rate variability, and deep sleep duration may be correlated with increases in the antibody response of the subject to the SARS-COV-2 spike protein.

In certain embodiments, the method further comprises measuring average overnight dermal temperature of the subject during the baseline period prior to vaccination of the subject to determine an average baseline value for the overnight dermal temperature, wherein the temperature deviation is computed as a difference between an average overnight temperature of the subject and the average baseline value of the overnight temperature.

In certain embodiments, vaccination of the subject comprises administering at least a first dose of the vaccine and a second dose of the vaccine to the subject. In some embodiments, one or more physiological metrics of the subject are measured before and after the second dose of the vaccine is administered to the subject.

In certain embodiments, vaccination comprises administering the vaccine according to a prime-boost regimen. For example, one or more booster doses of the vaccine may be administered to the subject. In some embodiments, one or more physiological metrics of the subject are measured before and after a booster dose of the vaccine is administered to the subject.

Individuals, who appear to have an insufficient antibody response, based on monitoring of their measured physiological metrics as described herein, may be advised by a clinician to avoid exposure to SARS-COV-2, e.g., by wearing an N95 mask around people and avoiding crowds as well as getting tested for SARS-COV-2 at the onset of any COVID-19 symptoms or known exposure to an individual who has COVID-19.

In certain embodiments, the method further comprises measuring a serum level of antibodies to the SARS-COV-2 spike protein RBD for the subject if the dermal temperature deviation, heart rate, respiratory rate, heart rate variability, and deep sleep duration on the first night after said vaccinating the subject indicate that the level of antibodies to the SARS-COV-2 spike protein RBD is below a threshold level for effective neutralization of SARS-COV-2. In some cases, another booster dose of the vaccine may be administered to the subject if the one or more physiological metrics of the subject indicate that the level of antibodies to the SARS-COV-2 spike protein RBD is below a threshold level for effective neutralization of SARS-COV-2.

### Systems

The present disclosure also provides a system which finds use in practicing the subject methods. The system is used for analyzing and processing physiological metrics, as described above, to determine whether vaccination of a subject against SARS-COV-2 produces an antibody response to the spike protein RBD. In some embodiments, the system may include: a) a measuring device configured to measure one or more physiological metrics of the subject (e.g., dermal temperature deviation, heart rate, respiratory rate, heart rate variability, and deep sleep duration) before and after vaccinating the subject against SARS-COV-2; b) a processor programmed to analyze the physiological metrics using one or more algorithms to correlate increases in the dermal temperature deviation, heart rate, and respiratory rate, and decreases in the heart rate variability and deep sleep duration on the first night after said vaccinating the subject compared to a pre-vaccination dermal temperature deviation, heart rate, respiratory rate, heart rate variability, and deep sleep duration with increases in the antibody response of the subject to the SARS-COV-2 spike protein RBD; and c) an output device configured to display information regarding the antibody response of the subject to the SARS-COV-2 spike protein RBD. In certain embodiments, the system further comprises a storage component operably coupled to the measuring device and the processor, wherein the storage component is configured to record physiological metric data measured by the measuring device.

In some embodiments, a computer implemented method is used for analyzing physiological metric data measured by the measuring device. The processor may be programmed to perform steps of the computer implemented method comprising: a) receiving the physiological metric data measured by the measuring device; b) analyzing the physiological metric data of the subject using one or more algorithms to correlate differences in physiological metric values measured on the first night after vaccinating the subject and pre-vaccination physiological metric values with increases in the antibody response of the subject to the SARS-COV-2 spike protein RBD; and c) displaying display information regarding the antibody response of the subject to the SARS-COV-2 spike protein RBD. Such information may include a determination of whether the subject is predicted to have an effective antibody response to the SARS-COV-2 spike protein RBD or an insufficient antibody response and may further provide a recommendation regarding administration of booster doses of the vaccine or another vaccine to the subject.

In certain embodiments, the computer implemented method further comprises storing a user profile for the subject comprising information regarding the physiological metric data acquired for the subject and the results of the analysis of the physiological metric data to determine whether vaccination of the subject against SARS-COV-2 produced an effective antibody response to the spike protein RBD of SARS-COV-2.

In certain embodiments, increases in the dermal temperature deviation, heart rate, and respiratory rate, and decreases in the heart rate variability and deep sleep duration on the first night following vaccination of the subject are correlated with increases in the antibody response to the SARS-COV-2 spike protein using a Spearman rank-order correlation analysis or a Kendall rank order correlation analysis.

The methods described herein can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, the system for performing the computer implemented method, as described, may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions. For example, the storage component includes instructions for analyzing the physiological metric data measured from the subject to determine whether vaccination of the subject against SARS-COV-2 produced an effective antibody response to the spike protein RBD of SARS-COV-2. The computer processor is coupled to the storage component and configured to execute the instructions stored in the storage component in order to receive physiological metric data of the subject and analyze the data according to one or more algorithms, as described herein. The display component displays information regarding the antibody response of the subject to the SARS-COV-2 spike protein RBD.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

Components of systems for carrying out the presently disclosed methods are further described in the examples below.

### Kits

Also provided are kits comprising a system and/or software for practicing the methods described herein. In some embodiments, the kit comprises a system for determining whether vaccination of a subject against SARS-COV-2 produces an antibody response to a spike protein RBD of the SARS-COV-2. Such a system may comprise: a) a measuring device configured to measure one or more physiological metrics of the subject selected from dermal temperature deviation, heart rate, respiratory rate, and heart rate variability before and after vaccinating the subject against SARS-COV-2; b) a processor programmed to analyze the physiological metrics using one or more algorithms to correlate increases in the dermal temperature deviation, heart rate, and respiratory rate, and decreases in the heart rate variability on the first night after said vaccinating the subject compared to a pre-vaccination dermal temperature deviation, heart rate, respiratory rate, and heart rate variability with increases in the antibody response of the subject to the SARS-COV-2 spike protein RBD; and c) an output device configured to display information regarding the antibody response of the subject to the SARS-COV-2 spike protein RBD.

The kit components may be present in packaging. In some instances, the assembled system may be contained in suitable packaging. In some instances, a kit may include the parts of the measuring device or components of the system in separate containers.

In addition to the above components, the subject kits may further include (in certain embodiments) instructions for practicing the subject methods using the kit. In some embodiments, instructions for assembling and/or using the system to obtain information regarding the antibody response of a subject to SARS-COV-2 after vaccination are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, SD drive, and the like, on which the information has been recorded. Yet another form of these instructions that may be present is a website address which may be used via the internet to access the information at a removed site.

### Utility

The methods, devices, and systems of the present disclosure find use in determining whether vaccination of a subject against severe acute respiratory syndrome coronavirus 2 (SARS-CoV-2) produces an antibody response to a spike protein receptor binding domain (RBD) of the SARS-COV-2. There is significant variability in neutralizing antibody responses to COVID-19 vaccines, which correlate with immune protection, but only limited information has been available about predictors of neutralizing antibody responses during vaccination. The disclosed methods correlate physiological information with levels of antibodies to the SARS-COV-2 receptor binding domain (RBD), the target of neutralizing antibodies generated by existing COVID-19 vaccines. Relevant physiological metrics such as dermal temperature deviation, heart rate, respiratory rate, heart rate variability, and deep sleep duration can be readily obtained from a wearable device worn by the subject before and after vaccination.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-31 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of determining whether vaccination of a subject against severe acute respiratory syndrome coronavirus 2 (SARS-COV-2) produces an antibody response to a spike protein receptor binding domain (RBD) of the SARS-COV-2, the method comprising:
vaccinating the subject by administering a vaccine against the SARS-COV-2; and
measuring one or more physiological metrics of the subject selected from dermal temperature deviation, heart rate, respiratory rate, heart rate variability, and deep sleep duration before and after said vaccinating the subject using a measuring device, wherein increases in the dermal temperature deviation, heart rate, and respiratory rate, and decreases in the heart rate variability and deep sleep duration on the first night after said vaccinating the subject compared to a pre-vaccination dermal temperature deviation, heart rate, respiratory rate, heart rate variability, and deep sleep duration of the subject correlate with increases in the antibody response of the subject to the SARS-COV-2 spike protein.

2. The method of aspect 1, wherein the vaccine comprises mRNA or DNA encoding the SARS-COV-2 spike protein RBD.

3. The method of aspect 1 or aspect 2, wherein the measuring comprises measuring two or more of the physiological metrics.

4. The method of aspect 3, wherein the measuring comprises measuring dermal temperature deviation, heart rate, respiratory rate, heart rate variability, and deep sleep duration.

5 The method of any one of aspects 1-4, wherein the measuring comprises measuring dermal temperature deviation.

6. The method of any one of aspects 1-5, wherein said vaccinating comprises administering at least a first dose of the vaccine and a second dose of the vaccine to the subject.

7. The method of aspect 6, wherein said measuring comprises measuring the one or more physiological metrics of the subject before and after the second dose of the vaccine is administered to the subject.

8. The method of aspect 6 or aspect 7, wherein said vaccinating comprises administering the vaccine according to a prime-boost regimen.

9 The method of aspect 8, wherein said vaccinating comprises administering one or more booster doses of the vaccine to the subject.

10. The method of aspect 9, wherein said measuring comprises measuring the one or more physiological metrics of the subject before and after a booster dose of the vaccine is administered to the subject.

11. The method of any one of aspects 1-10, wherein the measuring device is portable.

12. The method of any one of aspects 1-11, wherein the measuring device is a wearable device worn by the subject.

13. The method of any one of aspects 1-12, wherein the measuring device comprises a tri-axial accelerometer.

14. The method of any one of aspects 1-13, wherein heart rate, respiratory rate, and heart rate variability are measured from a photoplethysmogram (PPG) signal.

15. The method of any one of aspects 1-11, wherein dermal temperature is measured using a negative temperature coefficient (NTC) thermistor.

16. The method of aspect 15, wherein dermal temperature readings are acquired from the base of a finger on the palm side of a hand of the subject.

17. The method of any one of aspects 1-16, wherein the increases in the dermal temperature deviation, heart rate, and respiratory rate, and decreases in the heart rate variability and deep sleep duration on the first night following said vaccinating the subject are correlated with increases in the antibody response to the SARS-COV-2 spike protein using a Spearman rank-order correlation analysis or a Kendall rank order correlation analysis.

18. The method of any one of aspects 1-17, wherein the physiological metrics are measured during sleep periods, wake periods, or both sleep and wake periods of the subject.

19. The method of any one of aspects 1-18, further comprising measuring the one or more physiological metrics of the subject for at least one or two months prior to vaccination of the subject to determine average baseline values for the one or more physiological metrics, wherein increases in dermal temperature deviation, heart rate, and respiratory rate, and decreases in heart rate variability and deep sleep duration on the first night after said vaccinating the subject compared to the average baseline values for the dermal temperature deviation, heart rate, respiratory rate, heart rate variability, and deep sleep duration are correlated with increases in the antibody response of the subject to the SARS-COV-2 spike protein.

20. The method of aspect 19, further comprising measuring average overnight dermal temperature of the subject for at least one or two months prior to vaccination of the subject to determine an average baseline value for the overnight dermal temperature, wherein the temperature deviation is computed as a difference between an average overnight temperature of the subject and the average baseline value of the overnight temperature.

21. The method of aspect 19 or aspect 20, further comprising measuring a serum level of antibodies to the SARS-COV-2 spike protein RBD for the subject if the dermal temperature deviation, heart rate, respiratory rate, heart rate variability, and deep sleep duration on the first night after said vaccinating the subject indicate that the level of antibodies to the SARS-COV-2 spike protein RBD is below a threshold level for effective neutralization of SARS-COV-2.

22. The method of any one of aspects 1-21, further comprising administering another booster dose of the vaccine to the subject if the one or more physiological metrics of the subject are below a threshold level.

23. A system for determining whether vaccination of a subject against severe acute respiratory syndrome coronavirus 2 (SARS-COV-2) produces an antibody response to a spike protein receptor binding domain (RBD) of the SARS-COV-2, the system comprising:
a) a measuring device configured to measure one or more physiological metrics of the subject selected from dermal temperature deviation, heart rate, respiratory rate, heart rate variability, and deep sleep duration before and after vaccinating the subject against SARS-COV-2;
b) a processor programmed to analyze the physiological metrics using one or more algorithms to correlate increases in the dermal temperature deviation, heart rate, and respiratory rate, and decreases in the heart rate variability and deep sleep duration on the first night after said vaccinating the subject compared to a pre-vaccination dermal temperature deviation, heart rate, respiratory rate, heart rate variability, and deep sleep duration with increases in the antibody response of the subject to the SARS-COV-2 spike protein RBD; and
c) an output device configured to display information regarding the antibody response of the subject to the SARS-COV-2 spike protein RBD.

24. The system of aspect 23, further comprising a storage component operably coupled to the measuring device and the processor, wherein the storage component is configured to record physiological metric data measured by the measuring device.

25. The system of aspect 23 or 24, wherein the one or more algorithms are selected from a Spearman rank-order correlation analysis and a Kendall rank order correlation analysis.

26. The system of any one of aspects 23-25, wherein the measuring device comprises a negative temperature coefficient (NTC) thermistor.

27. The system of any one of aspects 23-26, wherein the measuring device comprises a tri-axial accelerometer.

28. The system of any one of aspects 23-27, wherein the measuring device measures heart rate, respiratory rate, and heart rate variability from a photoplethysmogram (PPG) signal.

29. The system of any one of aspects 23-28, wherein the measuring device is portable.

30. The system of any one of aspects 23-29, wherein the measuring device is a wearable device worn by the subject.

31. A kit comprising the system of any one of aspects 23-30, packaging for the system, and instructions for using the system for determining whether vaccination of a subject against severe acute respiratory syndrome coronavirus 2 (SARS-COV-2) produces an antibody response to a spike protein receptor binding domain (RBD) of the SARS-COV-2.

### EXAMPLES

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1: Metrics from Wearable Devices as Candidate Predictors of Antibody Response Following Vaccination Against COVID-19

### Introduction

Vaccines for COVID-19 have been remarkably effective in preventing severe disease, with reductions of the risk of severe disease in the 90% range (1,2). Existing COVID-19 vaccines do not eliminate the risk of severe disease, however, and there is also concern that protection may wane over time (3). The level of protection against COVID-19 has been shown to be strongly correlated with the level of antibodies directed at the SARS COV-2 viral spike protein-antibodies that are capable of viral neutralization (4). This raises two questions: (1) Are there modifiable factors that influence the level of antibodies generated by vaccination? (2) Are there elements of the physiological response to COVID-19 vaccination that are associated with greater antibody responses?

Prior data suggest that sleep duration just before and after vaccination is associated with the level of antibody responses to vaccination. For example, in an experimentally induced sleep restriction model, less sleep in the nights prior to influenza vaccination predicted lower responses to vaccination (5). Similarly, relative to a control group that had a normal night's sleep, participants whose sleep was restricted the night after receiving hepatitis A vaccination developed only half the antibody response (6). In the case of COVID-19 vaccination, this suggests that longer sleep duration prior to and just after vaccination might increase the likelihood of stronger antibody responses.

COVID-19 vaccinations result in physiological responses that are short in duration, but can be quite noticeable, including fever, chills, and fatigue (1,2). These physiological responses can differ considerably across individuals. The CDC website (7) on COVID-19 vaccination states that these possible side effects “are normal signs that your body is building protection.” This raises the question of whether greater physiological responses following vaccination might indicate that greater immune responses are developing. Limited data, however, have been published addressing this issue, and what data are available have not demonstrated self-reported side effects following COVID-19 vaccination to be associated with greater antibody responses (8).

We sought to shed light on these questions by analyzing data from the second TemPredict study, which took place in the spring of 2021, just as COVID-19 vaccinations were becoming widely available. In this study, we tested whether a wearable device (Oura Ring), which collects data on dermal temperature, heart rate, heart rate variability, and various sleep metrics (total sleep duration, deep sleep duration, and rapid eye movement (REM) sleep duration) could be used to detect the early period of COVID-19 disease. Most participants received a COVID-19 vaccine during the study. To assess antibody responses, we measured antibodies to the SARS COV-2 receptor binding domain (RBD) (9) at the end of the study period. Antibodies to the SARS COV-2 RBD correlate closely with viral neutralization, providing a simple serologic assay that is related to the level of immune protection achieved by vaccination (10). In the current analysis, we used physiological measures collected by the Oura Ring around the time of COVID-19 vaccination to assess possible predictors of antibody response as indexed by antibodies to the SARS COV-2 RBD.

### Methods

We initiated the second TemPredict study in December of 2020 to assess whether an algorithm derived from physiological metrics collected by an off-the-shelf wearable device (Oura Ring) could be used to detect COVID-19 infection in real time. An additional aim of this study was to assess whether data from this device could predict antibody response quantified as antibodies to SARS-COV-2 spike protein receptor binding domain (RBD), which is the focus of the current report.

### Study Participants

Recruitment. We recruited participants residing in the United States who already possessed Oura Rings by sending them email invitations. These email invitations included a link to an online consent survey. We also recruited participants who worked at participating sites (e.g., teachers, firefighters, and other first responders) by enlisting leadership at these sites to assist in recruitment. We mailed these sites recruitment materials, including study flyers and Oura Ring sizing kits, which contained plastic rings for prospective participants to try on to determine their size. We provided Oura Rings to interested individuals at these sites after they provided their size information to study coordinators.

Eligibility and Consent. Eligible participants were at least 18 years of age, possessed a smartphone that could pair with their Oura Ring, resided in the United States, did not previously have COVID-19 infection (verified through laboratory testing during enrollment), and could communicate in English. For this analysis, of the 2055 participants who completed the overall study, we first excluded participants who had a positive SARS COV-2 nucleocapsid antibody test at the end of the study, indicating COVID-19 infection during the study period (n=56). We then excluded participants who were not fully vaccinated at least 7 nights prior to their final blood draw (n=715). We then excluded participants who did not have at least 7 nights of physiological data within the timeframe used to develop the pre-vaccination baseline period (night-14 to night-4 prior to first vaccination or who lacked data for at least one night adjacent to vaccination; n=105).

The University of California San Francisco (UCSF) Institutional Review Board (IRB, IRB #20-30408) and the U.S. Department of Defense (DOD) Human Research Protections Office (HRPO, HRPO #E01877.1a) approved of all study activities, and all research was performed in accordance with relevant guidelines and regulations. All participants provided electronic (written) informed consent and this research was conducted according to the principles expressed in the Declaration of Helsinki. Participants to whom we provided Oura Rings kept the devices following their participation; we did not otherwise compensate participants for participation.

### Measures

Questionnaires: Beginning in December 2020, participants completed several online surveys. They first completed a baseline survey that collected demographic and health information. They also completed daily and monthly surveys on which they reported COVID-19 symptoms, COVID-19 diagnosis, and COVID-19 exposures. Within these surveys, they also reported whether they had been vaccinated against COVID-19, and if so, which vaccine they received (Pfizer-BioNTech, Moderna-NIAID, or Johnson & Johnson-Janssen) as well as their injection dates.

Antibody testing. We tested participants for antibodies to the SARS-COV-2 nucleocapsid protein (Test #164068, LabCorp, Inc.) during enrollment (December 2020 through early April 2021) and at the end of their participation (April and May 2021). The SARS COV-2 nucleocapsid antibody test becomes positive following COVID-19 infection but vaccination does not cause individuals to generate antibodies to this part of the virus. Participants were required to have a negative nucleocapsid antibody test at enrollment as we excluded participants with evidence of prior COVID-19 infection. At the end of the study period (late April and May 2021), we also tested participants for antibodies to the SARS-COV-2 RBD with the LabCorp Semi-Quantitative Total Antibody, Spike assay (Test #164090, LabCorp, Inc.). The dynamic range reported for this assay during the time when most of the study assays were performed was from 0.4 IU/ml to 2500 IU/ml, with a clinical cut off value for positive results of 0.8 U/mL. Prior to May 3, 2021, LabCorp reported results using an upper detection limit of 250 IU/ml, after which LabCorp changed assay procedures to quantitate antibody levels up to 2500 IU/ml. Fifty-seven participants completed the RBD antibody test before May 3, 2021, and had a result of “>250 IU/ml.”

Wearable device data (device-generated metrics). Participants wore the Oura Ring (Generation 2), a commercially available wearable sensor device (Oura Health, Oulu, Finland), on a finger of their choosing. The Oura Ring connects to the Oura App (available from the Google Play Store and the Apple App Store) via Bluetooth. Users can wear the ring continuously in both wet and dry environments.

The Oura Ring generates physiological metrics by aggregating data gathered from on-device sensors. These high-resolution metrics are transformed into summary metrics before transmission to a smartphone app. These device-generated metrics include nightly summary variables of dermal temperature deviations, resting heart rate (HR), resting heart rate variability (HRV), and respiratory rate (RR). The Oura Ring Gen2 assesses HR, HRV, and RR from a photoplethysmogram (PPG) signal generated at 250 Hz. The Oura ring calculates HR, HRV, and RR from inter-beat intervals (IBI), which the Oura Ring only generates during periods of sleep. The Oura Ring calculates HRV in the form of the root mean square of the successive differences (RMSSD). Tri-axial accelerometers estimate activity metrics as metabolic equivalents (MET) reported at 10-60 hz during both sleep and wake periods, and sleep stages at 5 min resolution. The Oura Ring assesses temperature by using a negative temperature coefficient (NTC) thermistor (resolution of 0.07° C.) on the internal surface of the ring. The sensor registers dermal temperature readings from the palm side of the finger base every 60 s. The temperature deviation metric is computed as the difference between a user's average overnight temperature and their longer-term baseline, which is calculated using a rolling window roughly equal to the prior two months. The Oura Ring also outputs sleep metrics that include minutes of light sleep (non-rapid eye movement [NREM] stages 1 and 2), deep sleep (NREM stages 3 and 4), rapid eye movement (REM) sleep, and total sleep time. We examined these metrics (temperature deviation, HR, HRV, RR, REM sleep duration, deep sleep duration, and total sleep duration) in the present analyses.

Vaccination. Participants reported the dates on which they received injections of one of the three vaccines available in the United States (Pfizer-BioNTech, Moderna-NIAID, or Johnson & Johnson-Janssen) between December 2020 and May 2021.

### Analytic Plan

Outcome. For correlation analyses, we treated values of “>2500 IU/ml” as 2500 IU/ml (n=474). We omitted participants who received a value of “>250 IU/ml;” n=51) from primary analyses, however, we report analyses including these values as 250 in supplementary analyses.

Predictors. We used device-generated values of each metric two nights before and three nights after each injection (nights −2,−1, 0, 1, 2, where 0 represents the night of the day when vaccination occurred). We established a pre-vaccination baseline period for each participant from 14 nights to 4 nights prior to the first vaccine injection (night-14 to night-4). We calculated values of each device-generated metric adjusting for this pre-vaccination baseline period by converting each physiological metric to a z-score using participants' respective individual means and standard deviations from the pre-vaccination baseline period. We examined device-generated metrics from all nights surrounding each injection (−2, −1, 0, 1, 2) and device-generated metrics adjusted for the pre-vaccination baseline period as predictors of RBD antibody responses. Notably, all device-generated metrics reflect values solely from the prior night except the temperature deviation metric (computed as the difference between the prior night's value as a deviation from an average derived of the prior two months). The temperature deviation metric adjusted for pre-vaccination baseline period therefore reflects a difference in two deviation metrics: the difference between a participant's (1) deviation on a particular night surrounding injection and (2) average deviation during the pre-vaccination baseline period.

If a participant was missing device-generated metrics from a particular night, we did not include them in analyses for that night. We excluded participants who had a positive SARS CoV-2 nucleocapsid antibody test at the end of the study, who did not receive their second injection (Moderna-NIAID and Pfizer-BioNTech) at least 7 nights prior to their final blood draw, who did not have at least 7 nights of physiological data within pre-vaccination baseline period (night-14 to night-4), and who had a threshold value of “>250 IU/ml” on the outcome.

Statistical analyses. We conducted analyses separately for each type of vaccine (Johnson & Johnson-Janssen, Moderna-NIAID, Pfizer-BioNTech). We also analyzed the data from both mRNA vaccines combined (Moderna-NIAID and Pfizer-BioNTech). First, we conducted Spearman rank-order correlations between RBD antibody responses and device-generated metrics, before and after adjusting for the pre-vaccination baseline period. We replicated these analyses using Kendall rank order correlation analyses (Kendall's tau-b) (11) because this approach directly accounts for tied ranks and better manages Type I error rates (12-14). We used these two approaches to evaluate whether these ordinal correlation analyses would yield a similar pattern of results

Second, we used results of the bivariate correlational analyses to inform variable selection for multivariate regression models that assessed which device-generated metrics independently predicted RBD antibody responses. Based on results from the Spearman correlations, we combined data from the mRNA vaccine recipients (Pfizer-BioNTech and Moderna-NIAID) from night 0 after the second injection to predict RBD antibody responses from device-generated metrics before and after adjusting for the pre-vaccination baseline period. We included device-generated metrics that had associations in Spearman correlation analyses (in the combined mRNA sample) before and/or after adjusting for the pre-vaccination baseline period with the RBD antibody responses with p-values<0.1. Due to the proportion of observations with right censored values of the outcome variable, we adopted a semi-parametric approach using Cox regression models (15), using the RBD antibody result as the dependent variable. Because these analyses used Cox regression to assess differences in antibody levels (rather than time to event typically used in Cox regression), we report coefficients rather than hazard ratios usually reported with Cox analyses. Coefficients offer insight into the direction and magnitude of associations between the RBD antibody responses and device-generated metrics (16). We found neither strong nor linear effects of time on antibody titer during the study period, and as a result, we did not include temporal parameters in the Cox regression models.

### Results

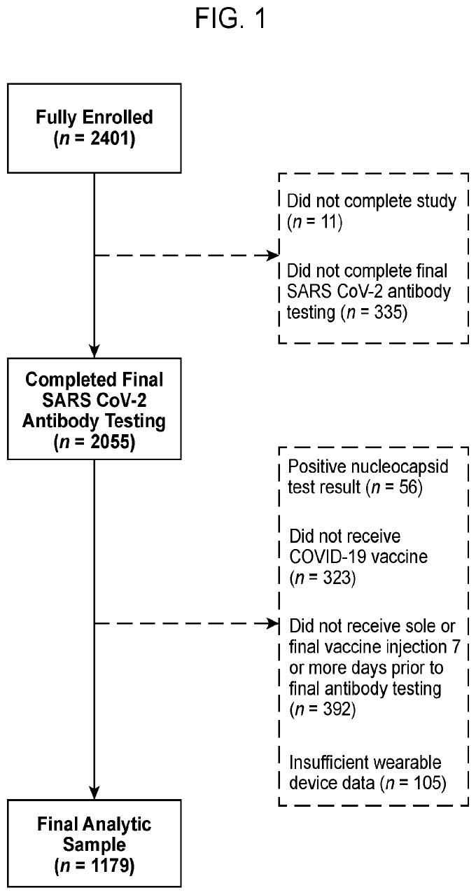

We enrolled 2,392 participants in the second TemPredict study (FIG. 1 and Table 1). After excluding participants who did not receive their second injection (Moderna-NIAID and Pfizer-BioNTech) at least 7 nights prior to their final blood draw, who did not have at least 7 nights of physiological data within the pre-vaccination baseline period (night-14 to night-4), who had a value of “>250 IU/ml” on the RBD antibody assay, or who had a positive value on their second nucleocapsid antibody test, there were 1,179 participants eligible for this analysis. Of these participants, 107 received Johnson & Johnson-Janssen COVID-19 vaccine, 366 received the Moderna-NIAID vaccine, and 706 received the Pfizer-BioNTech vaccine (Table 1). Of the 1179 participants included in this analysis, 474 had a result of “>2500 IU/ml” on the RBD antibody test, indicating substantial right censoring (40.2%) of the data. Four participants in the analysis dataset had a left censored RBD value (“<0.4 IU/ml”). Participants in the analytic sample obtained the RBD test an average of 38 days (SD: 30 days) after the final vaccine injection. By vaccine types, the mean (SD) days from final vaccination to RBD testing were: Moderna-NIAID 35 (24), Pfizer-BioNTech 40 (34), and Johnson & Johnson-Janssen 39 (14).

### Spearman Correlations

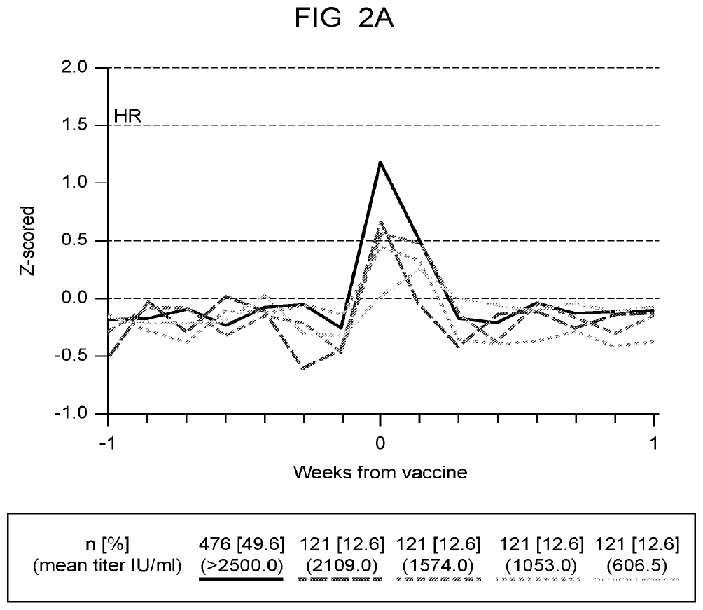

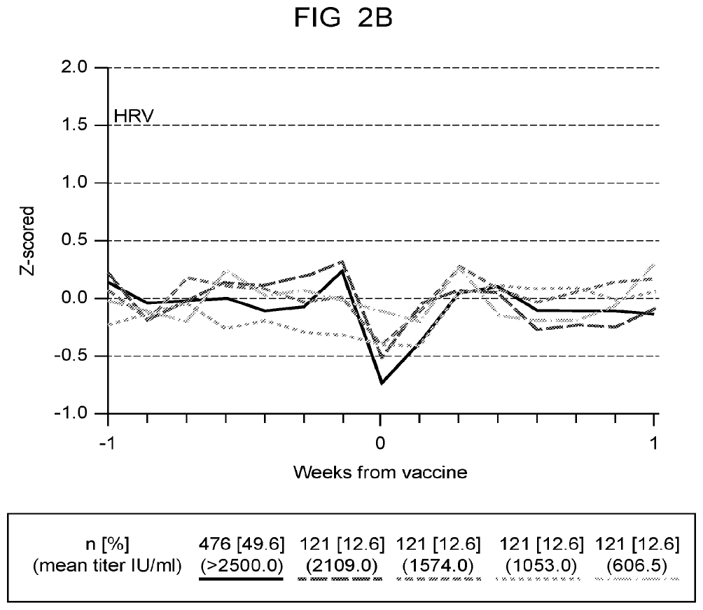

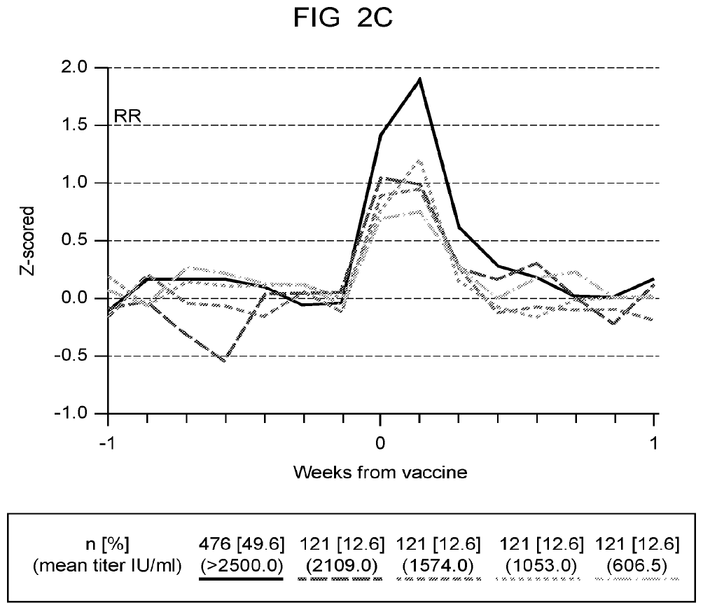

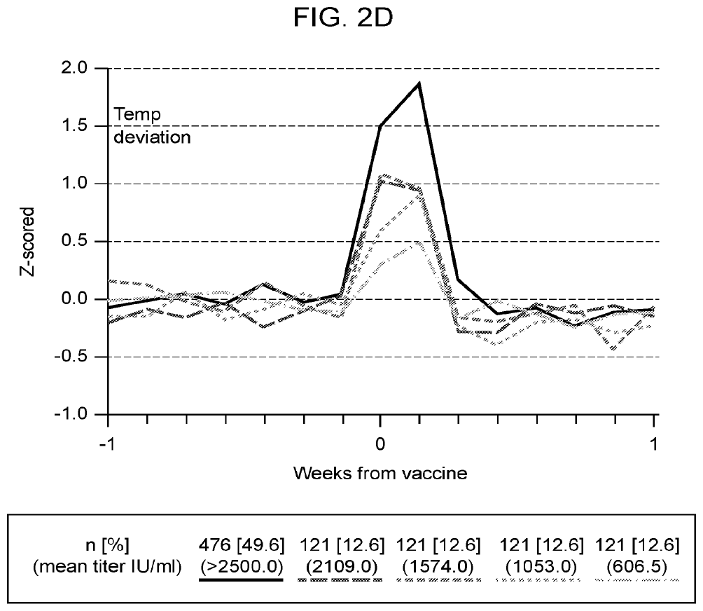

Changes in device-generated metrics during night 0 (the night immediately following second injection) tended to show a stronger pattern of associations with RBD antibody responses than these metrics the night immediately following the first injection (Table 2 and FIG. 2). In some cases, these associations were also evident the following night (night 1) after the second injection.

Device-generated metrics. Greater HR and temp deviation on night 0 after the second injection for both the Moderna-NIAID (HR: rho=0.138, p=0.012; temp deviation: rho=0.123, p=0.026) and Pfizer-BioNTech (HR: rho=0.176, p<. 001; temp deviation: rho=0.152, p<. 001) were associated with greater RBD antibody responses (Table 2). Additionally, on night 0 after the second injection, lower HRV values were associated with greater RBD antibody responses for Pfizer-BioNTech (HRV: rho=0.092, p=0.022), and these associations were in the same direction, but not statistically significant, for Moderna-NIAID (HRV: rho=0.056, p=0.312). The associations between RBD antibody responses and HR (Pfizer-BioNTech only) and temp deviation (Pfizer-BioNTech and Moderna-NAIAD) were also evident the following night (night 1) after the second injection. Correlations between device-generated metrics and antibody responses were in the same direction and often had similar rho values for participants who received the Johnson & Johnson-Janssen vaccine, but none of these correlations were statistically significant in the smaller group that received this vaccine.

Analyzing the two mRNA vaccines in combination (Moderna-NIAID and Pfizer-BioNTech), yielded similar associations between device-generated metrics and RBD antibody responses (Table 3). HR, temp deviation, and HRV from night 0 after the second injection were significantly associated with RBD antibody responses (HR: rho-. 197, p<. 001; temp deviation: rho=0.238, p<. 001; HRV: rho=0.118, p<. 001). In analyses combining participants who received either of the two mRNA vaccines, there was a statistically significant inverse correlation between deep sleep on night 0 after the second injection and RBD antibody responses (Deep: rho=0.079, p=0.014). The associations between RBD antibody responses and HR, RR, and temp deviation were also evident the following night (night 1) after the second injection.

Replicating these analyses retaining participants whose RBD antibody responses were “>250 IU/ml” as 250 IU/ml showed similar patterns of results (Supplementary Tables 1 and 2). Repeating these analyses using Kendall rank order correlation coefficients demonstrated similar patterns of results (Supplementary Tables 4 and 5).

Device-generated metrics adjusted for pre-vaccination baseline period. Adjusting for the pre-vaccination baseline period strengthened associations between device-generated metrics and RBD antibody responses revealed significant associations between RBD antibody values and additional metrics (Table 4). Specifically, greater increases in HR and temp deviation on night 0 after the second injection adjusted for the pre-vaccination baseline period for both Moderna-NIAID (HR: rho=0.148, p=0.007; temp deviation: rho-. 158, p=0.004) and Pfizer-BioNTech (HR: rho=0.124, p=0.002; temp deviation: rho=0.152, p<. 001) were associated with greater RBD antibody responses. Additionally, on night 0 after the second injection, larger decreases in HRV and deep sleep, and a larger increase in RR, were associated with greater RBD antibody responses for Moderna-NIAID (HRV: rho=0.119, p=0.031; RR: rho=0.139, p=0.012) and Pfizer-BioNTech (HRV: rho=−. 182, p<. 001; RR: rho=0.114, p=0.004). Pfizer-BioNTech also demonstrated an additional association between larger decreases in deep sleep and greater RBD antibody responses (Deep: rho=0.120, p=0.003). The associations between RBD antibody responses and both RR and temp deviation for each Pfizer-BioNTech and Moderna-NAIAD were also evident the following night (night 1) after the second injection. We did not observe these patterns for Johnson & Johnson-Janssen.

When we combined participants who received either of the two mRNA vaccines (Moderna-NIAID and Pfizer-BioNTech) the associations between device-generated metrics adjusted for pre-vaccination baseline and RBD antibody responses demonstrated greater statistical significance (Table 3). HR, HRV, RR, temp deviation, and deep sleep from night 0 after the second injection were significantly associated with RBD antibody responses. The associations between RBD antibody responses and HRV, HR, RR, and temp deviation were also statistically significant the following night (night 1) after the second injection. Additionally, greater HRV the night prior to the second injection (night-1) was significantly associated with greater RBD antibody responses (rho=0.07, p=0.024).

Replicating these analyses retaining participants whose RBD antibody responses were “>250 IU/ml” as 250 IU/ml showed similar patterns of results (Supplementary Tables 2 and 3). Repeating these analyses using Kendall rank order correlation coefficients demonstrated similar patterns of results (Supplementary Tables 5 and 6).

### Multivariate Models

Based on the results of bivariate analyses, we focused multivariate analysis on night 0 after the second vaccine injection, using the combined mRNA vaccine (Pfizer-BioNTech and Moderna-NIAID) participants. In Cox regression models, temp deviation on night 0 after the second injection was a statistically significant predictor of RBD antibody responses in models before and after adjusting for the pre-vaccination baseline period (Table 5). In the model unadjusted for the pre-vaccination baseline period, greater HR on night 0 after the second injection was also a statistically significantly predictor of greater RBD antibody responses.

### Discussion

We found that physiological metrics from an off-the-shelf wearable device on the two nights following the second dose of an mRNA-based COVID-19 vaccine were associated with RBD antibody responses. Using the device-generated metrics adjusted for the pre-vaccination baseline period, we found that both increased temperature deviation and heart rate (HR) and decreased heart rate variability (HRV) the night immediately following the second mRNA vaccine injection correlated with higher RBD antibody responses. In bivariate analyses using a standardized difference from the pre-vaccination baseline period for each physiological metric, we found that increased HR, temperature deviation, and RR as well as decreased HRV and deep sleep were each associated with higher RBD antibody responses for individuals who received the mRNA vaccines.

In a multivariate model predicting RBD antibody responses from device-generated metrics, we found that increased HR and temperature deviation independently predicted greater RBD antibody values for individuals who received the mRNA vaccines. In an identical model adjusting for the pre-vaccination baseline period, we found that dermal temperature was the sole statistically significant independent predictor of greater RBD antibody values in this same sample. Prior research has focused on identifying the roles of behavioral and psychological factors, in particular, sleep behaviors, in vaccine responses. Short sleep duration prior to vaccination against influenza reduces antibody responses in both observational and experimental sleep deprivation models (5,17). These observations have driven hypotheses that a longer sleep duration prior to vaccination against COVID-19 might boost host immune responses (18). Ongoing research is studying the effects of shift work and short sleep on antibody response following mRNA-based COVID-19 vaccination (19). Our data did not demonstrate associations between pre-vaccination sleep duration and antibody response among individuals receiving mRNA-based COVID-19 vaccines. In contrast to prior findings with other vaccines, we found that less deep sleep (NREM stages ¾ sleep) the night immediately after receiving an mRNA-based vaccine against COVID-19, both in absolute terms and relative to one's pre-vaccination levels, was associated with greater antibody responses. Rather than implicating reduced deep sleep after vaccination as a mechanism driving antibody response, a more likely explanation is that individuals experiencing more noticeable discomfort, such as arm pain, fever, chills, or other symptoms that can follow COVID-19 vaccination (7) may have experienced sleep disruptions. Consistent with this explanation, sleep duration was not a significant predictor of vaccine responses in multivariate models. Future research should capture diversified self-reports of the effects of post-vaccination symptomology, including their perceived effects on sleep factors (e.g., duration, restfulness) to further explore this hypothesis.

Mediators of systemic inflammatory responses, such as COX-2, are associated with fever as well as with the development of certain vaccine responses (20). As antipyretic analgesics, including acetaminophen (20) and non-steroidal anti-inflammatory drugs can inhibit COX-2, these data have raised concerns that antipyretic analgesics might blunt certain vaccine responses if given at the time of vaccination. Randomized, controlled trial results in children have shown that prophylactic administration of antipyretic drugs at the time of vaccination led to significantly lower antibody responses to multiple vaccines (22). Consistent with these data, the CDC recommends not taking antipyretic analgesics before COVID-19 vaccination (23). It is also possible that use of antipyretic analgesics following vaccination may blunt immune responses, however, few randomized controlled trials have formally examined this issue (20). The CDC website suggests that one “talk to a doctor about taking over-the-counter medicine, such as ibuprofen, acetaminophen, aspirin (only for people age 18 or older), or antihistamines for any pain and discomfort experienced after getting vaccinated” (7,23). To date, no trials that we are aware of have examined the role of fever or the impact of antipyretic medications on antibody responses following vaccination with mRNA vaccines. Although our data raise potential concerns that antipyretic analgesics might blunt COVID-19 vaccine responses, as temperature elevation was associated with greater antibody responses, our data do not directly address the effect of these medications, and do not shed light on whether antipyretics influence immune pathways involved in the generation of immune responses to COVID-19 vaccines. Taken together, prior research (20) and our data highlight the potential importance of further research testing the effects of antipyretic medications used after receiving COVID-19 vaccines on antibody responses.

Psychological factors, including psychological stress, can impact immunological responses to vaccination, and clarifying their impact on COVID-19 vaccination may be important for developing interventions to optimize antibody response (24). For example, prior research demonstrated the negative effects of stress on immune response following vaccination against Hepatitis B (24). More recent work has shown negative effects of poor sleep prior to vaccination against influenza (18), and subsequent studies have replicated similar patterns across multiple types of vaccinations (24). Decreased HRV can indicate increased sympathetic nervous system tone. Researchers have thus operationalized psychological stress using measures of heart rate variability (HRV; 23), although the correlation of heart rate variability with stress is imperfect and can depend on several moderators, including contextual factors (27). We found that in the combined mRNA vaccine group, on the night immediately prior to the second injection adjusted for baseline (night-1), HRV was positively correlated with antibody responses. This suggests that lower HRV (consistent with greater psychological stress (26,27)) was associated with lower antibody responses. This association changed direction in the following night (night 0) such that HRV was negatively correlated with antibody responses. This likely represents the effect of increased systemic inflammatory responses to vaccination, resulting in elevated temperature and HR, and decreased HRV, which in turn was associated with greater antibody responses. Consistent with this explanation, HRV did not significantly predict antibody response in multivariate models with other metrics (i.e., temperature) however, suggesting HRV after vaccination was not independently associated with antibody responses. Future research on this issue should measure stress more broadly (both self-report and physiological metrics of psychological stress) as predictors of antibody response to better clarify the role of stress in COVID-19 vaccine responses.

Our data have several limitations. We did not collect information on antipyretic or other medication use surrounding the time of vaccine injections, and thus cannot directly assess any effects of antipyretics on vaccine responses. We also did not collect detailed self-report information on post-vaccination symptomology. The RBD antibody assay we used had an upper limit of dynamic range of 2,500 IU/ml. A substantial proportion of participants achieved antibody levels above this range, resulting in right-censored data. To address this, we used Cox regression, a robust approach for analyzing right-censored data (readers may be more familiar with this approach when analyzing time-to-event data). Future research would benefit from an antibody assay with an extended dynamic range. We used a commercially available RBD antibody assay to assess neutralizing antibody responses rather than more precise but more expensive approaches such as pseudovirus neutralization assays. Like other studies exploring vaccination responses, our results will become more meaningful once there are enough data to form a scientific consensus on an antibody level that indicates adequate immune protection.

Conclusions. In conclusion, we found that several physiological metrics generated by an off-the-shelf wearable device in the nights following a second mRNA vaccination against COVID-19 were associated with subsequent levels of RBD antibody levels. In multivariate analyses, we found that elevated temperature was the strongest independent predictor of antibody responses. These findings suggest that off-the-shelf wearable devices collect data that could be useful in predicting immune responses to COVID-19 vaccination, though the clinical implications remain uncertain. Our data suggest that further investigation of the effects of antipyretics on COVID-19 vaccine responses is warranted.

### REFERENCES

1. Baden L R, El Sahly H M, Essink B, Kotloff K, Frey S, Novak R, et al. Efficacy and safety of the mRNA-1273 SARS-COV-2 vaccine. New England Journal of Medicine. 2021 Feb. 4; 384 (5): 403-16.
2. Heath P T, Galiza E P, Baxter D N, Boffito M, Browne D, Burns F, et al. Safety and efficacy of NVX-CoV2373 Covid-19 vaccine. New England Journal of Medicine. 2021 Sep. 23; 385 (13): 1172-83.
3. Pouwels K B, Pritchard E, Matthews P C, Stoesser N, Eyre D W, Vihta K-D, et al. Effect of Delta variant on viral burden and vaccine effectiveness against new SARS-COV-2 infections in the UK. Nat Med. 2021-10-14; 1-9.
4. Feng S, Phillips D J, White T, Sayal H, Aley P K, Bibi S, et al. Correlates of protection against symptomatic and asymptomatic SARS-COV-2 infection. Nat Med. 2021 November; 27 (11): 2032-40.
5. Spiegel K, Sheridan J F, Van Cauter E. Effect of sleep deprivation on response to immunizaton. JAMA. 2002-9-25; 288 (12): 1471-2.
6. Lange T, Perras B, Fehm H L, Born J. Sleep Enhances the Human Antibody Response to Hepatitis A Vaccination. Psychosomatic Medicine. 2003 October; 65 (5): 831-5.
7. CDC. What to Expect after Getting a COVID-19 Vaccine [Internet]. Centers for Disease Control and Prevention. 2021 [cited 2021-10-6]. Available from: https://www.cdc.gov/coronavirus/2019-ncov/vaccines/expect/after.html
8. Morales-Núñez JJ, Muñoz-Valle J F, Meza-López C, Wang L-F, Machado Sulbarán AC, Torres-Hernández P C, et al. Neutralizing antibodies titers and side effects in response to BNT162b2 vaccine in healthcare workers with and without prior SARS-COV-2 infection. Vaccines. 2021 July; 9 (7): 742.
9. 164090: SARS-COV-2 Semi-Quantitative Total Antibody, Spike|Labcorp [Internet]. [cited 2021-10-6]. Available from: https://www.labcorp.com/tests/164090/sars-cov-2-semi-quantitative-total-antibody-spike
10. Peterhoff D, Glück V, Vogel M, Schuster P, Schütz A, Neubert P, et al. A highly specific and sensitive serological assay detects SARS-COV-2 antibody levels in COVID-19 patients that correlate with neutralization. Infection. 2021-2-1; 49 (1): 75-82.
11. scipy.stats.kendalltau-SciPy v1.7.1 Manual [Internet]. [cited 2021 Nov. 22]. Available from: docs.scipy.org/doc/scipy/reference/generated/scipy.stats.kendalltau.html
12. Arndt S, Turvey C, Andreasen N C. Correlating and predicting psychiatric symptom ratings: Spearmans r versus Kendalls tau correlation. Journal of Psychiatric Research. 1999; 33 (2): 97-104.
13. Kendall M G. Rank correlation methods. United Kingdom: Griffin; 1948.
14. Kendall M G. A new measure of rank correlation. Biometrika. 1938; 30 (1/2): 81-93.
15. Vittinghoff E, McCulloch C E. Relaxing the rule of ten events per variable in logistic and Cox regression. Am J Epidemiol. 2007-3-15; 165 (6): 710-8.
16. The Cox Model|SpringerLink [Internet]. [cited 2021-11-20]. Available from: https://link.springer.com/chapter/10.1007% 2F978-1-4757-3294-8_3
17. Prather A A, Pressman S D, Miller G E, Cohen S. Links between self-reported sleep and antibody responses to the influenza vaccine. IntJ Behav Med. 2021 Feb. 1; 28 (1): 151-8.
18. Benedict C, Cedernaes J. Could a good night's sleep improve COVID-19 vaccine efficacy? The Lancet Respiratory Medicine. 2021-5-1; 9 (5): 447-8.
19. Lammers-van der Holst H M, Lammers G J, van der Horst G T J, Chaves I, de Vries R D, GeurtsvanKessel C H, et al. Understanding the association between sleep, shift work and COVID-19 vaccine immune response efficacy: Protocol of the S-CORE study. Journal of Sleep Research. n/a (n/a): e13496.
20. Saleh E, Moody M A, Walter E B. Effect of antipyretic analgesics on immune responses to vaccination. Human Vaccines & Immunotherapeutics. 2016 Sep. 1; 12 (9): 2391-402.
21. Hinz B, Cheremina O, Brune K. Acetaminophen (paracetamol) is a selective cyclooxygenase-2 inhibitor in man. FASEB J. 2008 February; 22 (2): 383-90.
22. Prymula R, Siegrist C-A, Chlibek R, Zemlickova H, Vackova M, Smetana J, et al. Effect of prophylactic paracetamol administration at time of vaccination on febrile reactions and antibody responses in children: Two open-label, randomised controlled trials. The Lancet. 2009-10-17; 374 (9698): 1339-50.
23. CDC. COVID-19 Vaccination [Internet]. Centers for Disease Control and Prevention. 2020 [cited 2021-11-15]. Available from: https://www.cdc.gov/coronavirus/2019-ncov/vaccines/prepare-for-vaccination.html
24. Madison A A, Shrout M R, Renna M E, Kiecolt-Glaser J K. Psychological and behavioral predictors of vaccine efficacy: Considerations for COVID-19. Perspect Psychol Sci. 2021-3-1; 16 (2): 191-203.
25. Glaser R, Kiecolt-Glaser J K, Bonneau R H, Malarkey W, Kennedy S, Hughes J. Stress-induced modulation of the immune response to recombinant hepatitis B vaccine. Psychosomatic Medicine. 1992; 54 (1): 22-9.
26. Kim H-G, Cheon E-J, Bai D-S, Lee Y H, Koo B-H. Stress and Heart Rate Variability: A Meta-Analysis and Review of the Literature. Psychiatry Investig. 2018 March; 15 (3): 235-45.
27. Thayer J F, Åhs F, Fredrikson M, Sollers J J, Wager T D. A meta-analysis of heart rate variability and neuroimaging studies: Implications for heart rate variability as a marker of stress and health. Neuroscience & Biobehavioral Reviews. 2012-2-1; 36 (2): 747-56.

TABLE 1
Participant characteristics.

| | | | | | Johnson & | | | |
|---|---|---|---|---|---|---|---|---|
| | Pfizer- | | Moderna- | | Johnson- | | | |
| | BioNTech | | NIAID | | Janssen | | Overall | |
| N | 706 | | 366 | | 107 | | 1179 | |
| Age (M, SD) | 50.4 | (11.4) | 52.8 | (12.0) | 49.5 | (9.9) | 51.0 | (11.5) |
| Biological sex (N, %) | | | | | | | | |
| Male | 334 | (47.3) | 166 | (45.4) | 53 | (49.5) | 553 | (46.9) |
| Female | 371 | (52.5) | 200 | (54.6) | 54 | (50.5) | 625 | (53.0) |
| Intersex | 1 | (0.1) | 0 | (0) | 0 | (0) | 1 | (0.1) |
| Race (N, %) | | | | | | | | |
| African | 1 | (0.1) | 1 | (0.3) | 0 | (0) | 2 | (0.2) |
| African American | 9 | (1.3) | 6 | (1.6) | 2 | (1.9) | 17 | (1.4) |
| Caribbean | 1 | (0.1) | 1 | (0.3) | 0 | (0) | 2 | (0.2) |
| | 603 | (85.4) | 325 | (88.8) | 95 | (88.8) | 1023 | (86.8) |
| Caucasian/White | 37 | (5.2) | 9 | (2.5) | 4 | (3.7) | 50 | (4.2) |
| East Asian | 5 | (0.7) | 3 | (0.8) | 1 | (0.9) | 9 | (0.8) |
| Middle Eastern | 3 | (0.4) | 0 | (0) | 0 | (0) | 3 | (0.3) |
| Native American/ Native Alaskan | 0 | (0) | 0 | (0) | 1 | (0.9) | 1 | (0.1) |
| Native Hawaiian / Other Pacific Islander | 16 | (2.3) | 3 | (0.8) | 1 | (0.9) | 20 | (1.7) |
| South Asian | 24 | (3.4) | 15 | (4.1) | 2 | (1.9) | 41 | (3.5) |
| More than 1 race | 7 | (1.0) | 3 | (0.8) | 1 | (0.9) | 11 | (0.9) |
| Prefer not to answer / Unknown | | | | | | | | |
| Hispanic/Latinx (N, %) | | | | | | | | |
| Yes | 30 | (4.2) | 17 | (4.6) | 7 | (6.5) | 54 | (4.6) |
| No | 671 | (95.0) | 347 | (94.8) | 100 | (93.5) | 1118 | (94.8) |
| Don't Know / Not Sure | 4 | (0.6) | 1 | (0.3) | 0 | (0) | 5 | (0.4) |
| Prefer not to answer | 1 | (0.1) | 1 | (0.3) | 0 | (0) | 2 | (0.2) |
| Education (N, %) | | | | | | | | |
| Less than a high school diploma | 0 | (0) | 0 | (0) | 0 | (0) | 0 | (0) |
| High school diploma or GED | 5 | (0.7) | 3 | (0.8) | 3 | (2.8) | 11 | (0.9) |
| Some college | 40 | (5.7) | 27 | (7.4) | 10 | (9.3) | 77 | (6.5) |
| Associate Degree (e.g., AA, AS) | 27 | (3.8) | 13 | (3.6) | 6 | (5.6) | 46 | (3.9) |
| Bachelor's Degree (e.g., BA, BS) | 270 | (38.2) | 131 | (35.8) | 40 | (37.4) | 441 | (37.4) |
| Master's Degree (e.g., MA, MS) | 223 | (31.6) | 119 | (32.5) | 38 | (35.5) | 380 | (32.2) |
| Advanced Degree (e.g., PhD, EdD, MD, | 141 | (20.0) | 73 | (19.9) | 10 | (9.3) | 224 | (19.0) |
| JD) | | | | | | | | |
| RBD Value (N, %) | | | | | | | | |
| Value of 0 to 2499 IU/ml | 458 | (64.9) | 89 | (24.3) | 107 | (100.0) | 654 | (55.5) |
| Value of “>250 IU/ml” | 33 | (4.7) | 18 | (4.9) | 0 | (0) | 51 | (4.3) |
| Value of “>2500 IU/ml” | 215 | (30.5) | 259 | (70.8) | 0 | (0) | 474 | (40.2) |

| RBD Value (Median, IQR) | 1613.0 | 2500.0 | 19.1 | 1956.5 |
|---|---|---|---|---|
| Value of 0 to 2499 IU/ml | [778.4, 2500.0] | [2477.0, 2500.0] | [6.1, 50.1] | [753.3, 2500.0] |
| Primary analysis data* | | | | |

Note.
IQR = Interquartile Range.
*Primary analysis data include values of “>2500 IU/ml” as 2500 IU/ml, excluding values of “>250 IU/ml.”

TABLE 2
Spearman rank order correlations between RBD antibody responses and
device-generated metrics on nights surrounding each injection.

| | | | Injection 1 | | Injection 1 | | | | Injection 2 | | | |
|---|---|---|---|---|---|---|---|---|---|---|---|---|
| | | | J&J | | Moderna | | Pfizer | | Moderna | | Pfizer | |
| | | Metric | rho | P | rho | P | rho | P | rho | P | rho | P |
| Night | −2 | Sleep Duration | −.127 | .207 | −.031 | .571 | .018 | .649 | .069 | .218 | −.023 | .564 |
| Relative | | REM Sleep | .026 | .799 | −.036 | .518 | .104 | .009 | .008 | .886 | .061 | .126 |
| to | | Deep Sleep | −.139 | .166 | −.024 | .657 | .010 | .808 | .070 | .214 | .049 | .219 |
| Injection | | HRV (RMSSD) | −.119 | .236 | .013 | .812 | −.046 | .251 | .007 | .908 | .025 | .536 |
| | | HR | −.038 | .706 | .033 | .547 | .138 | .000 | .031 | .583 | .063 | .117 |
| | | RR | .072 | .477 | −.048 | .381 | −.067 | .092 | −.130 | .020 | −.045 | .260 |
| | | Temp Deviation | −.028 | .784 | −.012 | .830 | .052 | .193 | .043 | .439 | −.005 | .891 |
| | −1 | Sleep Duration | .036 | .723 | −.022 | .683 | −.044 | .270 | −.059 | .289 | −.068 | .088 |
| | | REM Sleep | .018 | .856 | −.062 | .255 | .033 | .403 | −.086 | .126 | .001 | .976 |
| | | Deep Sleep | .012 | .908 | −.048 | .375 | .047 | .236 | .005 | .933 | .048 | .235 |
| | | HRV (RMSSD) | −.081 | .421 | .049 | .366 | .001 | .974 | .028 | .614 | .044 | .275 |
| | | HR | .056 | .574 | .021 | .704 | .103 | .009 | .020 | .726 | .093 | .020 |
| | | RR | .122 | .223 | −.047 | .387 | −.094 | .018 | −.048 | .391 | −.079 | .048 |
| | | Temp Deviation | −.047 | .640 | −.023 | .670 | .002 | .951 | .053 | .342 | −.023 | .568 |
| | 0 | Sleep Duration | .125 | .211 | .066 | .235 | −.080 | .044 | −.003 | .960 | −.033 | .408 |
| | | REM Sleep | .054 | .593 | .045 | .413 | −.002 | .968 | .024 | .659 | .033 | .407 |
| | | Deep Sleep | −.162 | .106 | −.010 | .860 | .005 | .899 | −.025 | .654 | −.058 | .148 |
| | | HRV (RMSSD) | −.047 | .638 | .011 | .838 | .006 | .873 | −.056 | .312 | −.092 | .022 |
| | | HR | .125 | .213 | .008 | .885 | .101 | .010 | .138 | .012 | .176 | .000 |
| | | RR | .123 | .222 | −.053 | .340 | −.058 | .141 | −.021 | .711 | −.004 | .925 |
| | | Temp Deviation | .155 | .122 | −.009 | .870 | −.003 | .935 | .123 | .026 | .152 | .000 |
| | 1 | Sleep Duration | −.202 | .044 | −.053 | .334 | −.002 | .957 | .069 | .221 | −.009 | .823 |
| | | REM Sleep | −.056 | .580 | .023 | .674 | .045 | .258 | .068 | .231 | .082 | .041 |
| | | Deep Sleep | −.149 | .138 | −.006 | .912 | .042 | .290 | −.090 | .110 | .026 | .522 |
| | | HRV (RMSSD) | −.073 | .471 | .003 | .962 | −.012 | .765 | .011 | .842 | −.016 | .696 |
| | | HR | .049 | .631 | .002 | .968 | .087 | .028 | .053 | .352 | .116 | .004 |
| | | RR | .157 | .119 | −.030 | .585 | −.066 | .098 | −.007 | .899 | .049 | .229 |
| | | Temp Deviation | .093 | .356 | .055 | .316 | −.030 | .451 | .136 | .016 | .186 | .000 |
| | 2 | Sleep Duration | .046 | .646 | −.019 | .735 | −.016 | .684 | −.061 | .282 | −.106 | .008 |
| | | REM Sleep | .019 | .853 | −.026 | .641 | .032 | .425 | −.038 | .497 | −.001 | .974 |
| | | Deep Sleep | .026 | .795 | −.039 | .478 | −.006 | .870 | .012 | .832 | −.013 | .739 |
| | | HRV (RMSSD) | −.074 | .465 | .038 | .491 | .005 | .891 | .004 | .942 | −.041 | .312 |
| | | HR | .108 | .282 | .033 | .552 | .089 | .025 | .041 | .467 | .100 | .013 |
| | | RR | .144 | .152 | .000 | .997 | −.090 | .023 | −.048 | .394 | −.027 | .496 |
| | | Temp Deviation | .187 | .062 | .001 | .987 | −.019 | .641 | .081 | .153 | .021 | .594 |

Note.
See Methods for variable descriptions. rho = Spearman's Rank Order Correlation Coefficient; Night relative to injection = −2 as 2 nights before vaccine, −1 as one night before injection, 0 as night immediately after injection, 1 as night the day after injection, 2 as 2 nights after injection; Temp Deviation = Temperature deviation; REM = Rapid Eye Movement; Deep = Stages Non-REM 3 and 4 sleep; HRV = Heart Rate Variability; RMSSD = Root Mean Square of Successive Differences; HR = Heart Rate; RR = Respiratory Rate.

TABLE 3
Spearman rank order correlations between RBD antibody responses and
device-generated metrics before and after adjusting for the pre-
vaccination baseline period on nights surrounding injections for
Moderna-NIAID and Pfizer-BioNTech vaccine recipients, combined.

| | | | Device-generated Metric | | | | Adjusted by Baseline period | | | |
|---|---|---|---|---|---|---|---|---|---|---|
| | | | Injection 1 | | Injection 2 | | Injection 1 | | Injection 2 | |
| | | Metric | rho | P | rho | P | rho | P | rho | P |
| Night | −2 | Sleep Duration | −.001 | .982 | .021 | .519 | .001 | .988 | .062 | .057 |
| Relative | | REM Sleep | .056 | .079 | .059 | .071 | .028 | .380 | .038 | .245 |
| to | | Deep Sleep | −.008 | .808 | .042 | .194 | −.004 | .901 | .040 | .221 |
| Injection | | HRV (RMSSD) | −.031 | .338 | −.007 | .819 | −.065 | .043 | .004 | .904 |
| | | HR | .100 | .002 | .051 | .120 | .081 | .012 | −.022 | .496 |
| | | RR | −.041 | .197 | −.071 | .030 | .058 | .071 | −.011 | .735 |
| | | Temp Deviation | .017 | .603 | .001 | .967 | .044 | .167 | .023 | .488 |
| | −1 | Sleep Duration | −.011 | .729 | −.042 | .199 | −.010 | .764 | −.023 | .488 |
| | | REM Sleep | .036 | .264 | −.015 | .648 | .008 | .798 | −.050 | .123 |
| | | Deep Sleep | .017 | .594 | .031 | .340 | .056 | .079 | .024 | .463 |
| | | HRV (RMSSD) | −.001 | .981 | .043 | .186 | .011 | .741 | .074 | .024 |
| | | HR | .070 | .029 | .052 | .114 | −.009 | .773 | −.011 | .746 |
| | | RR | −.056 | .080 | −.064 | .048 | .009 | .770 | −.026 | .430 |
| | | Temp Deviation | .008 | .813 | −.015 | .644 | .036 | .264 | .018 | .591 |
| | 0 | Sleep Duration | −.023 | .476 | −.012 | .704 | −.018 | .565 | .024 | .462 |
| | | REM Sleep | .010 | .755 | .031 | .343 | −.024 | .460 | .022 | .497 |
| | | Deep Sleep | −.025 | .430 | −.079 | .014 | −.014 | .667 | −.126 | .000 |
| | | HRV (RMSSD) | −.010 | .749 | −.118 | .000 | −.007 | .816 | −.199 | .000 |
| | | HR | .070 | .029 | .197 | .000 | .013 | .695 | .208 | .000 |
| | | RR | −.039 | .221 | .038 | .241 | .069 | .030 | .177 | .000 |
| | | Temp Deviation | .006 | .850 | .238 | .000 | .023 | .471 | .244 | .000 |
| | 1 | Sleep Duration | −.018 | .573 | .065 | .047 | −.016 | .625 | .125 | .000 |
| | | REM Sleep | .034 | .289 | .081 | .014 | .010 | .757 | .084 | .011 |
| | | Deep Sleep | .014 | .655 | −.012 | .710 | .028 | .381 | −.021 | .522 |
| | | HRV (RMSSD) | −.032 | .317 | −.029 | .385 | −.080 | .013 | −.074 | .024 |
| | | HR | .072 | .024 | .100 | .002 | .035 | .281 | .083 | .012 |
| | | RR | −.015 | .643 | .067 | .042 | .097 | .002 | .260 | .000 |
| | | Temp Deviation | .040 | .215 | .241 | .000 | .065 | .042 | .250 | .000 |
| | 2 | Sleep Duration | .012 | .714 | −.084 | .010 | .031 | .329 | −.083 | .011 |
| | | REM Sleep | .037 | .252 | −.013 | .690 | .031 | .327 | −.048 | .146 |
| | | Deep Sleep | −.024 | .462 | −.020 | .533 | −.018 | .584 | −.031 | .350 |
| | | HRV (RMSSD) | −.001 | .980 | −.031 | .337 | .008 | .799 | −.051 | .120 |
| | | HR | .068 | .034 | .068 | .038 | .024 | .457 | .005 | .871 |
| | | RR | −.051 | .111 | −.008 | .816 | .054 | .092 | .117 | .000 |
| | | Temp Deviation | .013 | .693 | .068 | .039 | .021 | .507 | .076 | .020 |

Note.
See Table 2 note. Pre-vaccination baseline period taken from nights −14 to −4 prior to first injection, see Methods.

TABLE 4
Spearman rank order correlations between RBD antibody responses and device-generated
metrics nights 0, 1, and 2, adjusted for the pre-vaccination baseline period.

| | | | Injection 1 | | Injection 1 | | | | Injection 2 | | | |
|---|---|---|---|---|---|---|---|---|---|---|---|---|
| | | | J&J | | Moderna | | Pfizer | | Moderna | | Pfizer | |
| | | Metric | rho | P | rho | P | rho | P | rho | P | rho | P |
| Night | 0 | Sleep Duration | .166 | .098 | .097 | .079 | −.050 | .207 | .015 | .784 | .022 | .589 |
| Relative | | REM Sleep | .106 | .293 | .051 | .355 | −.044 | .269 | .037 | .501 | .012 | .763 |
| to | | Deep Sleep | −.173 | .083 | −.010 | .863 | −.009 | .816 | −.054 | .326 | −.120 | .003 |
| Injection | | HRV (RMSSD) | −.001 | .993 | −.045 | .422 | .012 | .758 | −.119 | .031 | −.182 | .000 |
| | | HR | .125 | .212 | −.026 | .636 | −.018 | .642 | .148 | .007 | .124 | .002 |
| | | RR | .113 | .262 | .008 | .881 | .074 | .061 | .139 | .012 | .114 | .004 |
| | | Temp Deviation | .141 | .160 | .021 | .710 | .000 | .994 | .158 | .004 | .152 | .000 |
| | 1 | Sleep Duration | −.224 | .025 | −.068 | .215 | .026 | .508 | .088 | .120 | .081 | .045 |
| | | REM Sleep | −.281 | .005 | .058 | .292 | −.009 | .818 | .112 | .048 | .065 | .109 |
| | | Deep Sleep | −.152 | .130 | .024 | .667 | .047 | .238 | −.123 | .030 | .005 | .892 |
| | | HRV (RMSSD) | .002 | .986 | −.042 | .439 | −.060 | .131 | −.004 | .946 | −.058 | .147 |
| | | HR | .042 | .677 | −.049 | .376 | −.005 | .896 | .042 | .460 | .054 | .182 |
| | | RR | .057 | .573 | .103 | .061 | .041 | .299 | .168 | .003 | .259 | .000 |
| | | Temp Deviation | .067 | .511 | .094 | .085 | −.009 | .814 | .169 | .003 | .182 | .000 |
| | 2 | Sleep Duration | .030 | .769 | −.006 | .912 | .034 | .391 | −.066 | .246 | −.064 | .111 |
| | | REM Sleep | −.125 | .214 | −.018 | .740 | .016 | .685 | −.080 | .155 | −.031 | .446 |
| | | Deep Sleep | .052 | .602 | −.018 | .738 | −.013 | .742 | .046 | .415 | −.065 | .106 |
| | | HRV (RMSSD) | −.039 | .701 | .076 | .164 | −.014 | .717 | .004 | .937 | −.112 | .005 |
| | | HR | .111 | .269 | .010 | .858 | .021 | .602 | .003 | .963 | .033 | .408 |
| | | RR | .082 | .416 | .099 | .069 | .028 | .477 | .138 | .014 | .097 | .016 |
| | | Temp Deviation | .195 | .051 | .044 | .422 | −.023 | .561 | .124 | .027 | .009 | .821 |

Note.
See Table 2 and Table 3 notes for variable descriptions and preparations.

TABLE 5
Multivariate regression models predicting RBD antibody responses from device-
generated metrics before and after adjusting for the pre-vaccination baseline
period that demonstrated associations with RBD antibody responses in Spearman
correlations from night 0 after the second injection for Moderna-NIAID
and Pfizer-BioNTech vaccine recipients, combined.

| | Coefficient | | | |
|---|---|---|---|---|
| | (Beta) | 95% CI (LB, UB) | Z | p |
| Metric | | | | |
| GFI −log2(p) = 44.95 | | | | |
| HRV (RMSSD) | −0.002 | (−0.007, 0.004) | −0.584 | .559 |
| RR | 0.035 | (−0.024, 0.093) | 1.161 | .246 |
| HR | −0.019 | (−0.031, −0.007) | −3.178 | .002 |
| Deep | 0.000 | (0.000, 0.000) | 1.125 | .260 |
| Temp Deviation | −0.515 | (−0.707, −0.322) | −5.247 | <.001 |
| Metric Adjusted by Baseline period | | | | |
| GFI -log2(p) = 44.65 | | | | |
| HRV (RMSSD) | 0.047 | (−0.023, 0.117) | 1.308 | 191 |
| RR | −0.042 | (−0.091, 0.008) | −1.632 | 103 |
| HR | −0.013 | (−0.073, 0.047) | −0.428 | .669 |
| Deep | 0.036 | (−0.032, 0.104) | 1.043 | .297 |
| Temp Deviation | −0.071 | (−0.109, −0.034) | −3.748 | <.001 |

Note.
See Table 2 and Table 3 notes for variable descriptions and preparations. In Cox regression models, coefficient values are inverted from the usual linear regression interpretation: Negative coefficients represent decreased hazard, which in this context translates to increased RBD antibody responses. Positive coefficients represent increased hazard, which in this context translates to decreased antibody levels. Because we used antibody titer in place of survival time in these models, coefficients represent association between the change in the predictor and the rank of the RBD antibody value.

SUPPLEMENTARY TABLE 1
Spearman rank order correlations between RBD antibody responses and device-generated metrics
(retaining n = 51 values of “>250 IU/ml” as 250 IU/ml) on nights surrounding each injection.

| | | | Injection 1 | | Injection 1 | | | | Injection 2 | | | |
|---|---|---|---|---|---|---|---|---|---|---|---|---|
| | | | J&J | | Moderna | | Pfizer | | Moderna | | Pfizer | |
| | | Metric | rho | P | rho | P | rho | P | rho | P | rho | P |
| Night | −2 | Sleep Duration | −.127 | .207 | −.012 | .819 | .019 | .617 | .027 | .618 | −.025 | .528 |
| Relative | | REM Sleep | .026 | .799 | −.028 | .596 | .095 | .015 | −.015 | .787 | .041 | .297 |
| to | | Deep Sleep | −.139 | .166 | .004 | .938 | .040 | .298 | .100 | .066 | .063 | .109 |
| Injection | | HRV (RMSSD) | −.119 | .236 | .039 | .467 | −.053 | .172 | .010 | .861 | .010 | .800 |
| | | HR | −.038 | .706 | .028 | .595 | .149 | .000 | .022 | .684 | .085 | .030 |
| | | RR | .072 | .477 | −.073 | .170 | −.037 | .336 | −.128 | .018 | −.022 | .575 |
| | | Temp Deviation | −.028 | .784 | −.064 | .234 | .061 | .115 | .011 | .841 | −.010 | .789 |
| | −1 | Sleep Duration | .036 | .723 | −.017 | .756 | −.050 | .193 | −.014 | .796 | −.044 | .266 |
| | | REM Sleep | .018 | .856 | −.058 | .278 | .012 | .753 | −.055 | .314 | .004 | .915 |
| | | Deep Sleep | .012 | .908 | −.017 | .746 | .069 | .073 | .012 | .826 | .057 | .145 |
| | | HRV (RMSSD) | −.081 | .421 | .074 | .163 | −.004 | .922 | .060 | .270 | .028 | .480 |
| | | HR | .056 | .574 | −.001 | .987 | .112 | .004 | .000 | .998 | .114 | .003 |
| | | RR | .122 | .223 | −.061 | .251 | −.060 | .119 | −.072 | .185 | −.054 | .168 |
| | | Temp Deviation | −.047 | .640 | −.046 | .390 | −.017 | .654 | .054 | .325 | −.009 | .820 |
| | 0 | Sleep Duration | .125 | .211 | .096 | .076 | −.082 | .034 | .001 | .991 | −.027 | .482 |
| | | REM Sleep | .054 | .593 | .045 | .400 | .002 | .952 | .054 | .321 | .027 | .487 |
| | | Deep Sleep | −.162 | .106 | −.006 | .913 | .025 | .520 | −.022 | .680 | −.029 | .452 |
| | | HRV (RMSSD) | −.047 | .638 | .037 | .494 | −.005 | .892 | −.066 | .222 | −.090 | .020 |
| | | HR | .125 | .213 | .012 | .829 | .104 | .007 | .148 | .006 | .185 | .000 |
| | | RR | .123 | .222 | −.059 | .275 | −.034 | .380 | .000 | .996 | .025 | .531 |
| | | Temp Deviation | .155 | .122 | .006 | .917 | −.021 | .587 | .132 | .014 | .133 | .001 |
| | 1 | Sleep Duration | −.202 | .044 | −.044 | .408 | .001 | .980 | .042 | .448 | .004 | .919 |
| | | REM Sleep | −.056 | .580 | .028 | .600 | .045 | .252 | .045 | .417 | .082 | .038 |
| | | Deep Sleep | −.149 | .138 | −.006 | .913 | .061 | .114 | −.051 | .350 | .043 | .272 |
| | | HRV (RMSSD) | −.073 | .471 | .025 | .639 | −.024 | .530 | .039 | .481 | −.026 | .514 |
| | | HR | .049 | .631 | .005 | .931 | .103 | .008 | .030 | .587 | .132 | .001 |
| | | RR | .157 | .119 | −.036 | .505 | −.043 | .268 | −.035 | .525 | .073 | .065 |
| | | Temp Deviation | .093 | .356 | .045 | .400 | −.040 | .302 | .096 | .081 | .172 | .000 |
| | 2 | Sleep Duration | .046 | .646 | .008 | .884 | −.004 | .925 | −.003 | .959 | −.091 | .020 |
| | | REM Sleep | .019 | .853 | .002 | .970 | .036 | .350 | .008 | .879 | −.004 | .921 |
| | | Deep Sleep | .026 | .795 | −.018 | .732 | .006 | .878 | .044 | .424 | −.002 | .955 |
| | | HRV (RMSSD) | −.074 | .465 | .026 | .631 | −.021 | .592 | .034 | .539 | −.044 | .263 |
| | | HR | .108 | .282 | .038 | .483 | .109 | .005 | .024 | .659 | .110 | .005 |
| | | RR | .144 | .152 | −.036 | .497 | −.055 | .156 | −.065 | .233 | −.006 | .880 |
| | | Temp Deviation | .187 | .062 | .006 | .912 | .000 | .997 | .099 | .072 | .003 | .940 |

Note.
See Table 2 note. Analyses are identical to Table 2, however, retain the n = 51 participants whose RBD antibody responses were returned from LabCorp as “>250 IU/ml” as having values of 250 IU/ml.

SUPPLEMENTARY TABLE 2
Spearman rank order correlations between RBD antibody responses and device-generated
metrics before and after adjusting for the pre-vaccination baseline period (retaining
n = 51 values of “>250 IU/ml” as 250 IU/ml) on nights surrounding
injections for Moderna-NIAID and Pfizer-BioNTech vaccine recipients, combined.

| | | | Device-generated Metric | | | | Adjusted by Baseline period | | | |
|---|---|---|---|---|---|---|---|---|---|---|
| | | | Injection 1 | | Injection 2 | | Injection 1 | | Injection 2 | |
| | | Metric | rho | P | rho | P | rho | P | rho | P |
| Night | −2 | Sleep Duration | .005 | .868 | .008 | .802 | .008 | .800 | .038 | .230 |
| Relative | | REM Sleep | .052 | .100 | .036 | .252 | .018 | .559 | .014 | .656 |
| to | | Deep Sleep | .022 | .487 | .062 | .051 | .010 | .756 | .045 | .157 |
| Injection | | HRV (RMSSD) | −.028 | .370 | −.015 | .638 | −.058 | .064 | −.003 | .915 |
| | | HR | .107 | .001 | .063 | .046 | .079 | .012 | −.029 | .367 |
| | | RR | −.030 | .335 | −.055 | .080 | .058 | .064 | −.018 | .566 |
| | | Temp Deviation | .010 | .753 | −.010 | .758 | .034 | .278 | .013 | .676 |
| | −1 | Sleep Duration | −.017 | .587 | −.014 | .659 | −.029 | .358 | −.001 | .976 |
| | | REM Sleep | .019 | .551 | −.007 | .835 | −.013 | .670 | −.041 | .202 |
| | | Deep Sleep | .041 | .188 | .040 | .208 | .052 | .095 | .022 | .481 |
| | | HRV (RMSSD) | .005 | .878 | .040 | .212 | .024 | .438 | .073 | .022 |
| | | HR | .070 | .025 | .063 | .048 | −.035 | .268 | −.018 | .573 |
| | | RR | −.040 | .205 | −.053 | .093 | −.003 | .935 | −.033 | .299 |
| | | Temp Deviation | −.014 | .658 | −.003 | .916 | .015 | .627 | .027 | .390 |
| | 0 | Sleep Duration | −.017 | .590 | −.008 | .792 | −.022 | .479 | .014 | .648 |
| | | REM Sleep | .013 | .668 | .035 | .271 | −.023 | .463 | .017 | .591 |
| | | Deep Sleep | −.007 | .814 | −.056 | .074 | −.022 | .491 | −.114 | .000 |
| | | HRV (RMSSD) | −.010 | .754 | −.117 | .000 | .006 | .849 | −.188 | .000 |
| | | HR | .072 | .022 | .203 | .000 | −.009 | .785 | .195 | .000 |
| | | RR | −.026 | .403 | .058 | .065 | .061 | .052 | .183 | .000 |
| | | Temp Deviation | −.004 | .899 | .221 | .000 | .014 | .650 | .228 | .000 |
| | 1 | Sleep Duration | −.014 | .647 | .061 | .057 | −.008 | .796 | .109 | .001 |
| | | REM Sleep | .036 | .253 | .073 | .022 | .005 | .863 | .067 | .035 |
| | | Deep Sleep | .029 | .359 | .010 | .752 | .020 | .525 | −.020 | .541 |
| | | HRV (RMSSD) | −.032 | .301 | −.025 | .428 | −.076 | .015 | −.059 | .064 |
| | | HR | .082 | .009 | .105 | .001 | .031 | .325 | .067 | .037 |
| | | RR | −.005 | .876 | .071 | .026 | .095 | .002 | .228 | .000 |
| | | Temp Deviation | .026 | .407 | .211 | .000 | .049 | .121 | .219 | .000 |
| | 2 | Sleep Duration | .025 | .425 | −.059 | .065 | .039 | .208 | −.062 | .051 |
| | | REM Sleep | .045 | .151 | −.002 | .948 | .033 | .299 | −.034 | .286 |
| | | Deep Sleep | −.009 | .773 | −.002 | .956 | −.009 | .765 | −.023 | .467 |
| | | HRV (RMSSD) | −.021 | .510 | −.025 | .427 | −.009 | .775 | −.026 | .409 |
| | | HR | .083 | .008 | .072 | .024 | .012 | .704 | −.017 | .591 |
| | | RR | −.038 | .228 | −.001 | .980 | .039 | .215 | .095 | .003 |
| | | Temp Deviation | .025 | .430 | .058 | .070 | .031 | .321 | .061 | .056 |

Note
See Table 2 and Table 3 notes for variable descriptions and preparations. Analyses are identical to Table 3, however, retain the n = 51 participants whose RBD antibody responses were returned from LabCorp as “>250 IU/ml” as having values of 250 IU/ml.

SUPPLEMENTARY TABLE 3
Spearman rank order correlations between RBD antibody responses and device-
generated metrics (retaining n = 51 values of “>250 IU/ml”
as 250 IU/ml) on nights 0, 1, and 2, adjusted for the pre-vaccination baseline period.

| | | | Injection 1 | | Injection 1 | | | | Injection 2 | | | |
|---|---|---|---|---|---|---|---|---|---|---|---|---|
| | | | J&J | | Moderna | | Pfizer | | Moderna | | Pfizer | |
| | | Metric | rho | P | rho | P | rho | P | rho | P | rho | P |
| Night | 0 | Sleep Duration | .166 | .098 | .094 | .083 | −.057 | .138 | .011 | .841 | .011 | .784 |
| Relative | | REM Sleep | .106 | .293 | .025 | .641 | −.035 | .367 | .038 | .488 | .005 | .899 |
| to | | Deep Sleep | −.173 | .083 | −.036 | .510 | −.011 | .779 | −.072 | .181 | −.100 | .011 |
| [symbol] | | HRV (RMSSD) | −.001 | .993 | −.014 | .791 | .020 | .605 | −.158 | .003 | −.155 | .000 |
| | | HR | .125 | .212 | −.027 | .620 | −.042 | .276 | .158 | .003 | .111 | .004 |
| | | RR | .113 | .262 | .035 | .516 | .054 | .160 | .181 | .001 | .112 | .004 |
| | | Temp Deviation | .141 | .160 | .026 | .624 | −.013 | .744 | .157 | .003 | .139 | .000 |
| | 1 | Sleep Duration | −.224 | .025 | −.062 | .248 | .034 | .382 | .045 | .410 | .082 | .038 |
| | | REM Sleep | −.281 | .005 | .043 | .423 | −.010 | .798 | .066 | .228 | .059 | .138 |
| | | Deep Sleep | −.152 | .130 | −.026 | .630 | .054 | .164 | −.102 | .063 | .003 | .936 |
| | | HRV (RMSSD) | .002 | .986 | −.046 | .392 | −.057 | .146 | −.009 | .863 | −.040 | .309 |
| | | HR | .042 | .677 | −.031 | .565 | −.009 | .810 | .018 | .737 | .046 | .243 |
| | | RR | .057 | .573 | .108 | .043 | .041 | .290 | .126 | .022 | .233 | .000 |
| | | Temp Deviation | .067 | .511 | .085 | .113 | −.024 | .535 | .127 | .021 | .169 | .000 |
| | 2 | Sleep Duration | .030 | .769 | −.004 | .938 | .047 | .223 | −.037 | .505 | −.048 | .219 |
| | | REM Sleep | −.125 | .214 | −.019 | .728 | .022 | .575 | −.049 | .377 | −.022 | .569 |
| | | Deep Sleep | .052 | .602 | −.004 | .940 | −.008 | .841 | .043 | .437 | −.054 | .173 |
| | | HRV (RMSSD) | −.039 | .701 | .039 | .467 | −.026 | .498 | .018 | .738 | −.079 | .043 |
| | | HR | .111 | .269 | −.014 | .797 | .014 | .715 | −.034 | .542 | .013 | .749 |
| | | RR | .082 | .416 | .049 | .358 | .026 | .510 | .082 | .132 | .088 | .024 |
| | | Temp Deviation | .195 | .051 | .043 | .420 | −.006 | .878 | .117 | .032 | −.006 | .874 |

Note.
See Table 2 and Table 3 notes for variable descriptions and preparations. Analyses are identical to Table 4, however, retain the n = 51 participants whose RBD antibody responses were returned from LabCorp as “>250 IU/ml” (rather than a specific value). [symbol]
indicates data missing or illegible when filed

SUPPLEMENTARY TABLE 4
Kendall rank order correlations between RBD antibody responses and
device-generated metrics on nights surrounding each injection.

| | | | Injection 1 | | Injection 1 | | | | Injection 2 | | | |
|---|---|---|---|---|---|---|---|---|---|---|---|---|
| | | | J&J | | Moderna | | Pfizer | | Moderna | | Pfizer | |
| | | Metric | τ | P | τ | P | τ | P | τ | P | τ | P |
| Night | −2 | Sleep Duration | −.086 | .204 | −.024 | .574 | .011 | .692 | .053 | .219 | −.016 | .563 |
| Relative | | REM Sleep | .019 | .774 | −.029 | .494 | .073 | .008 | .007 | .866 | .043 | .124 |
| to | | Deep Sleep | −.095 | .162 | −.019 | .647 | .006 | .829 | .054 | .213 | .034 | .222 |
| Injection | | HRV (RMSSD) | −.076 | .262 | .012 | .782 | −.033 | .237 | .006 | .885 | .017 | .543 |
| | | HR | −.022 | .747 | .025 | .553 | .096 | .000 | .025 | .561 | .043 | .120 |
| | | RR | .053 | .436 | −.038 | .372 | −.048 | .082 | −.102 | .019 | −.033 | .243 |
| | | Temp Deviation | −.009 | .890 | −.009 | .833 | .037 | .186 | .033 | .446 | −.004 | .884 |
| | −1 | Sleep Duration | .025 | .716 | −.017 | .685 | −.031 | .261 | −.043 | .314 | −.048 | .086 |
| | | REM Sleep | .018 | .792 | −.048 | .253 | .022 | .418 | −.064 | .134 | .001 | .971 |
| | | Deep Sleep | .005 | .942 | −.036 | .392 | .032 | .242 | .005 | .904 | .033 | .241 |
| | | HRV (RMSSD) | −.054 | .425 | .038 | .363 | .000 | .996 | .021 | .627 | .031 | .267 |
| | | HR | .036 | .589 | .015 | .716 | .072 | .009 | .014 | .748 | .064 | .021 |
| | | RR | .085 | .211 | −.036 | .394 | −.067 | .016 | −.037 | .392 | −.056 | .044 |
| | | Temp Deviation | −.028 | .683 | −.017 | .688 | .002 | .955 | .041 | .344 | −.016 | .566 |
| | 0 | Sleep Duration | .084 | .213 | .049 | .246 | −.056 | .041 | −.003 | .940 | −.024 | .396 |
| | | REM Sleep | .033 | .628 | .036 | .399 | −.001 | .961 | .019 | .652 | .024 | .395 |
| | | Deep Sleep | −.111 | .100 | −.007 | .861 | .003 | .908 | −.019 | .654 | −.040 | .147 |
| | | HRV (RMSSD) | −.029 | .668 | .008 | .850 | .005 | .870 | −.044 | .308 | −.064 | .023 |
| | | HR | .085 | .209 | .006 | .889 | .070 | .011 | .105 | .014 | .123 | .000 |
| | | RR | .085 | .214 | −.039 | .362 | −.041 | .141 | −.016 | .707 | −.004 | .892 |
| | | Temp Deviation | .112 | .097 | −.008 | .857 | −.002 | .956 | .096 | .024 | .107 | .000 |
| | 1 | Sleep Duration | −.135 | .047 | −.041 | .330 | −.001 | .961 | .053 | .223 | −.005 | .864 |
| | | REM Sleep | −.035 | .606 | .016 | .699 | .030 | .278 | .054 | .215 | .057 | .040 |
| | | Deep Sleep | −.089 | .189 | −.004 | .919 | .029 | .298 | −.068 | .120 | .018 | .516 |
| | | HRV (RMSSD) | −.045 | .508 | .001 | .981 | −.009 | .754 | .009 | .835 | −.011 | .690 |
| | | HR | .036 | .598 | .001 | .976 | .061 | .028 | .040 | .359 | .080 | .004 |
| | | RR | .111 | .107 | −.024 | .576 | −.046 | .097 | −.005 | .909 | .034 | .228 |
| | | Temp Deviation | .066 | .334 | .042 | .316 | −.021 | .446 | .104 | .017 | .129 | .000 |
| | 2 | Sleep Duration | .032 | .639 | −.016 | .695 | −.012 | .660 | −.047 | .280 | −.075 | .007 |
| | | REM Sleep | .010 | .883 | −.020 | .635 | .021 | .438 | −.029 | .508 | −.001 | .977 |
| | | Deep Sleep | .022 | .740 | −.029 | .487 | −.005 | .867 | .008 | .845 | −.010 | .715 |
| | | HRV (RMSSD) | −.042 | .538 | .029 | .498 | .004 | .896 | .005 | .913 | −.029 | .307 |
| | | HR | .078 | .248 | .025 | .549 | .062 | .025 | .031 | .481 | .069 | .014 |
| | | RR | .098 | .153 | −.001 | .981 | −.065 | .020 | −.038 | .385 | −.021 | .453 |
| | | Temp Deviation | .130 | .055 | .000 | .998 | −.013 | .628 | .062 | .152 | .014 | .617 |

Note.
See Table 2 note.

SUPPLEMENTARY TABLE 5
Kendall rank order correlations between RBD antibody responses and device-generated metrics
before and after adjusting by the pre-vaccination baseline period on nights surrounding
injections for Moderna-NIAID and Pfizer-BioNTech vaccine recipients, combined.

| | | | Device-generated Metric | | | | Adjusted by Baseline period | | | |
|---|---|---|---|---|---|---|---|---|---|---|
| | | | Injection 1 | | Injection 2 | | Injection 1 | | Injection 2 | |
| | | Metric | τ | P | τ | P | τ | P | τ | P |
| Night | −2 | Sleep Duration | −.001 | .949 | .015 | .526 | — | — | — | — |
| Relative | | REM Sleep | .041 | .074 | .042 | .070 | — | — | — | — |
| to | | Deep Sleep | −.006 | .788 | .030 | .196 | — | — | — | — |
| Injection | | HRV (RMSSD) | −.022 | .334 | −.005 | .818 | — | — | — | — |
| | | HR | .072 | .002 | .036 | .119 | — | — | — | — |
| | | RR | −.031 | .183 | −.052 | .027 | | | | |
| | | Temp Deviation | .012 | .603 | .001 | .973 | — | — | — | — |
| | −1 | Sleep Duration | −.008 | .724 | −.030 | .201 | — | — | — | — |
| | | REM Sleep | .026 | .266 | −.011 | .644 | — | — | — | — |
| | | Deep Sleep | .012 | .596 | .022 | .343 | — | — | — | — |
| | | HRV (RMSSD) | −.001 | .972 | .031 | .188 | — | — | — | — |
| | | HR | .050 | .030 | .037 | .114 | — | — | — | — |
| | | RR | −.041 | .074 | −.047 | .046 | | | | |
| | | Temp Deviation | .005 | .815 | −.011 | .652 | — | — | — | — |
| | 0 | Sleep Duration | −.017 | .471 | −.009 | .692 | −.013 | .573 | .017 | .461 |
| | | REM Sleep | .007 | .762 | .023 | .332 | −.017 | .466 | .016 | .497 |
| | | Deep Sleep | −.019 | .420 | −.057 | .014 | −.010 | .656 | −.091 | .000 |
| | | HRV (RMSSD) | −.007 | .751 | −.085 | .000 | −.005 | .826 | −.143 | .000 |
| | | HR | .050 | .030 | .141 | .000 | .008 | .717 | .149 | .000 |
| | | RR | −.029 | .219 | .027 | .251 | .050 | .028 | .127 | .000 |
| | | Temp Deviation | .005 | .843 | .172 | .000 | .017 | .472 | .176 | .000 |
| | 1 | Sleep Duration | −.013 | .562 | .047 | .043 | −.011 | .638 | .090 | .000 |
| | | REM Sleep | .023 | .312 | .058 | .013 | .007 | .749 | .060 | .010 |
| | | Deep Sleep | .010 | .659 | −.009 | .711 | .020 | .377 | −.014 | .548 |
| | | HRV (RMSSD) | −.023 | .312 | −.020 | .388 | −.057 | .013 | −.054 | .022 |
| | | HR | .052 | .025 | .071 | .002 | .024 | .286 | .059 | .012 |
| | | RR | −.011 | .638 | .048 | .042 | .070 | .002 | .187 | .000 |
| | | Temp Deviation | .029 | .212 | .174 | .000 | .046 | .044 | .180 | .000 |
| | 2 | Sleep Duration | .007 | .766 | −.061 | .010 | .025 | .276 | −.060 | .010 |
| | | REM Sleep | .026 | .260 | −.009 | .695 | .022 | .334 | −.034 | .143 |
| | | Deep Sleep | −.017 | .457 | −.015 | .530 | −.013 | .586 | −.022 | .339 |
| | | HRV (RMSSD) | −.001 | .974 | −.023 | .332 | .005 | .812 | −.036 | .124 |
| | | HR | .048 | .035 | .048 | .040 | .017 | .470 | .004 | .873 |
| | | RR | −.038 | .102 | −.007 | .775 | .040 | .083 | .083 | .000 |
| | | Temp Deviation | .009 | .708 | .048 | .042 | .015 | .504 | .054 | .022 |

Note.
See Table 2 and Table 3 notes for variable descriptions and preparations.
Physiological metric pre-vaccination baseline period taken from nights −14 to −4 prior to first injection.

SUPPLEMENTARY TABLE 6
Kendall rank order correlations between RBD antibody responses and device-generated
metrics on nights 0, 1, and 2, adjusted for the pre-vaccination baseline period.

| | | | Injection 1 | | Injection 1 | | | | Injection 2 | | | |
|---|---|---|---|---|---|---|---|---|---|---|---|---|
| | | | J&J | | Moderna | | Pfizer | | Moderna | | Pfizer | |
| | | Metric | τ | P | τ | P | τ | P | τ | P | τ | P |
| Night | 0 | Sleep Duration | .108 | .110 | .073 | .088 | −.033 | .228 | .012 | .784 | .016 | .574 |
| Relative | | REM Sleep | .068 | .313 | .042 | .325 | −.030 | .271 | .028 | .511 | .009 | .753 |
| to | | Deep Sleep | −.119 | .077 | −.007 | .862 | −.007 | .802 | −.042 | .320 | −.084 | .002 |
| Injection | | HRV (RMSSD) | −.003 | .963 | −.034 | .419 | .010 | .728 | −.091 | .031 | −.126 | .000 |
| | | HR | .092 | .175 | −.020 | .637 | −.014 | .606 | .114 | .007 | .086 | .002 |
| | | RR | .077 | .252 | .006 | .886 | .052 | .059 | .108 | .011 | .079 | .004 |
| | | Temp Deviation | .113 | .096 | .016 | .710 | −.001 | .983 | .122 | .004 | .107 | .000 |
| | 1 | Sleep Duration | −.150 | .027 | −.053 | .208 | .018 | .512 | .068 | .116 | .056 | .044 |
| | | REM Sleep | −.186 | .006 | .044 | .297 | −.006 | .818 | .084 | .052 | .046 | .103 |
| | | Deep Sleep | −.115 | .090 | .019 | .652 | .032 | .240 | −.092 | .035 | .004 | .873 |
| | | HRV (RMSSD) | .001 | .990 | −.031 | .454 | −.041 | .132 | −.004 | .926 | −.042 | .137 |
| | | HR | .027 | .692 | −.038 | .368 | −.004 | .874 | .032 | .454 | .037 | .183 |
| | | RR | .043 | .522 | .079 | .060 | .030 | .283 | .127 | .003 | .180 | .000 |
| | | Temp Deviation | .055 | .416 | .072 | .087 | −.007 | .796 | .128 | .003 | .127 | .000 |
| | 2 | Sleep Duration | .022 | .747 | −.004 | .923 | .027 | .322 | −.050 | .251 | −.046 | .102 |
| | | REM Sleep | −.080 | .234 | −.015 | .722 | .011 | .698 | −.063 | .144 | −.021 | .453 |
| | | Deep Sleep | .025 | .716 | −.014 | .740 | −.009 | .734 | .035 | .412 | −.046 | .100 |
| | | HRV (RMSSD) | −.026 | .699 | .059 | .159 | −.011 | .687 | .004 | .921 | −.076 | .006 |
| | | HR | .074 | .272 | .008 | .855 | .014 | .614 | .001 | .981 | .023 | .406 |
| | | RR | .054 | .420 | .076 | .069 | .021 | .446 | .107 | .014 | .066 | .018 |
| | | Temp Deviation | .137 | .043 | .034 | .416 | −.016 | .556 | .096 | .027 | .005 | .848 |

Note.
See Table 2 and Table 3 notes for variable descriptions and preparations.

The above examples are provided to illustrate the invention but not to limit its scope. Other variants of the invention will be readily apparent to one of ordinary skill in the art and are encompassed by the appended claims. All publications, accessions, references, databases, and patents cited herein are hereby incorporated by reference for all purposes.

1. A method of determining whether vaccination of a subject against severe acute respiratory syndrome coronavirus 2 (SARS-COV-2) produces an antibody response to a spike protein receptor binding domain (RBD) of the SARS-COV-2, the method comprising:
vaccinating the subject by administering a vaccine against the SARS-COV-2; and
measuring one or more physiological metrics of the subject selected from dermal temperature deviation, heart rate, respiratory rate, heart rate variability, and deep sleep duration before and after said vaccinating the subject using a measuring device, wherein increases in the dermal temperature deviation, heart rate, and respiratory rate, and decreases in the heart rate variability and deep sleep duration on the first night after said vaccinating the subject compared to a pre-vaccination dermal temperature deviation, heart rate, respiratory rate, heart rate variability, and deep sleep duration of the subject correlate with increases in the antibody response of the subject to the SARS-COV-2 spike protein.

2. The method of claim 1, wherein the vaccine comprises mRNA or DNA encoding the SARS-COV-2 spike protein RBD.

3. The method of claim 1, wherein the measuring comprises measuring two or more of the physiological metrics.

4. The method of claim 3, wherein the measuring comprises measuring dermal temperature deviation, heart rate, respiratory rate, heart rate variability, and deep sleep duration.

5. (canceled)

6. The method of claim 1, wherein said vaccinating comprises administering at least a first dose of the vaccine and a second dose of the vaccine to the subject, wherein said measuring comprises measuring the one or more physiological metrics of the subject before and after the second dose of the vaccine is administered to the subject.

7. (canceled)

8. The method of claim 6, wherein said vaccinating comprises administering the vaccine according to a prime-boost regimen.

9. The method of claim 8, wherein said vaccinating comprises administering one or more booster doses of the vaccine to the subject.

10. The method of claim 9, wherein said measuring comprises measuring the one or more physiological metrics of the subject before and after a booster dose of the vaccine is administered to the subject.

11. The method of claim 1, wherein the measuring device is a portable device or a wearable device worn by the subject.

12. (canceled)

13. The method of claim 1, wherein the measuring device comprises a tri-axial accelerometer.

14. The method of claim 1, wherein the heart rate, respiratory rate, and heart rate variability are measured from a photoplethysmogram (PPG) signal, and wherein the dermal temperature is measured using a negative temperature coefficient (NTC) thermistor.

15-16. (canceled)

17. The method of claim 1, wherein the increases in the dermal temperature deviation, heart rate, and respiratory rate, and decreases in the heart rate variability and deep sleep duration on the first night following said vaccinating the subject are correlated with increases in the antibody response to the SARS-CoV-2 spike protein using a Spearman rank-order correlation analysis or a Kendall rank order correlation analysis.

18. The method of claim 1, wherein the physiological metrics are measured during sleep periods, wake periods, or both sleep and wake periods of the subject.

19. The method of claim 1, further comprising measuring the one or more physiological metrics of the subject for at least one or two months prior to vaccination of the subject to determine average baseline values for the one or more physiological metrics, wherein increases in dermal temperature deviation, heart rate, and respiratory rate, and decreases in heart rate variability and deep sleep duration on the first night after said vaccinating the subject compared to the average baseline values for the dermal temperature deviation, heart rate, respiratory rate, heart rate variability, and deep sleep duration are correlated with increases in the antibody response of the subject to the SARS-CoV-2 spike protein.

20. The method of claim 19, further comprising measuring average overnight dermal temperature of the subject for at least one or two months prior to vaccination of the subject to determine an average baseline value for the overnight dermal temperature, wherein the temperature deviation is computed as a difference between an average overnight temperature of the subject and the average baseline value of the overnight temperature; and/or
further comprising measuring a serum level of antibodies to the SARS-COV-2 spike protein RBD for the subject if the dermal temperature deviation, heart rate, respiratory rate, heart rate variability, and deep sleep duration on the first night after said vaccinating the subject indicate that the level of antibodies to the SARS-COV-2 spike protein RBD is below a threshold level for effective neutralization of SARS-COV-2.

21. (canceled)

22. The method of claim 1, further comprising administering another booster dose of the vaccine to the subject if the one or more physiological metrics of the subject indicate that the level of antibodies to the SARS-COV-2 spike protein RBD is below a threshold level for effective neutralization of SARS-COV-2.

23. A system for determining whether vaccination of a subject against severe acute respiratory syndrome coronavirus 2 (SARS-COV-2) produces an antibody response to a spike protein receptor binding domain (RBD) of the SARS-COV-2, the system comprising:
a) a measuring device configured to measure one or more physiological metrics of the subject selected from dermal temperature deviation, heart rate, respiratory rate, heart rate variability, and deep sleep duration before and after vaccinating the subject against SARS-COV-2;
b) a processor programmed to analyze the physiological metrics using one or more algorithms to correlate increases in the dermal temperature deviation, heart rate, and respiratory rate, and decreases in the heart rate variability and deep sleep duration on the first night after said vaccinating the subject compared to a pre-vaccination dermal temperature deviation, heart rate, respiratory rate, heart rate variability, and deep sleep duration with increases in the antibody response of the subject to the SARS-COV-2 spike protein RBD; and
c) an output device configured to display information regarding the antibody response of the subject to the SARS-COV-2 spike protein RBD.

24. The system of claim 23, further comprising a storage component operably coupled to the measuring device and the processor, wherein the storage component is configured to record physiological metric data measured by the measuring device.

25. The system of claim 23, wherein the one or more algorithms are selected from a Spearman rank-order correlation analysis and a Kendall rank order correlation analysis.

26. The system of claim 23, wherein the measuring device comprises a negative temperature coefficient (NTC) thermistor or a tri-axial accelerometer.

27. (canceled)

28. The system of claim 23, wherein the measuring device measures heart rate, respiratory rate, and heart rate variability from a photoplethysmogram (PPG) signal.

29. The system of claim 23, wherein the measuring device is a portable device or a wearable device worn by the subject.

30. (canceled)

31. A kit comprising the system of claim 23, packaging for the system, and instructions for using the system for determining whether vaccination of a subject against severe acute respiratory syndrome coronavirus 2 (SARS-COV-2) produces an antibody response to a spike protein receptor binding domain (RBD) of the SARS-COV-2.
